(19) United States
(12) Patent Application Publication
(10) Pub. No.: US 2011/0077396 A1
(43) Pub. Date: Mar. 31, 2011

(54) INTERMEDIATES FOR HYDROXYLATED CONTRAST ENHANCEMENT AGENTS

(75) Inventors: Brian James Grimmond, Clifton Park, NY (US); Michael James Rishel, Saratoga Springs, NY (US); Michael Todd Luttrell, Clifton Park, NY (US)

(73) Assignee: General Electric Company, Schenectady, NY (US)

(21) Appl. No.: 12/785,110

(22) Filed: May 21, 2010

Related U.S. Application Data
(63) Continuation-in-part of application No. 12/570,705, filed on Sep. 30, 2009.
Application No. 12/570,705 is a continuation-in-part of application No. 12/614,729, filed on Nov. 9, 2009.

(51) Int. Cl. C07F 15/02; C07D 319/08; C07C 229/36; C07F 7/18; C07C 215/20

(52) U.S. Cl. 544/64; 549/365; 562/443; 556/424; 564/360; 560/35; 556/418

(57) ABSTRACT

In one aspect, the present invention provides a protected ligand precursor having structure XX

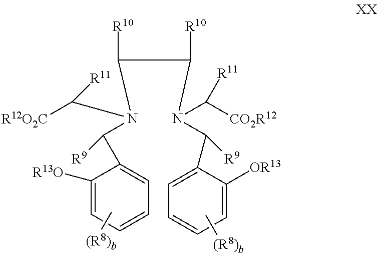

wherein R⁸ is independently at each occurrence a protected hydroxy group, a protected C₁-C₃ hydroxyalkyl group, or a C₁-C₃ alkyl group, and b is 0-4; R⁹-R¹¹ are independently at each occurrence hydrogen, a protected C₁-C₃ hydroxyalkyl group, or a C₁-C₃ alkyl group, with the proviso that at least one of R⁸-R¹¹ is a protected hydroxy group or a protected C₁-C₃ hydroxyalkyl group; and R¹² and R¹³ are independently at each occurrence a protecting group is selected from the group consisting of C₁-C₃₀ aliphatic radicals, C₃-C₃₀ cycloaliphatic radicals, and C₂-C₃₀ aromatic radicals.

### CROSS REFERENCE TO RELATED APPLICATIONS

This application is a continuation in part of and claims priority from co-pending United States patent application entitled “Hydroxylated Contrast Enhancement Agents” filed Sep. 30, 2009 and having a docket number 233560-1 and Ser. No. 12/570,705, and United States patent application entitled “Intermediates For Hydroxylated Contrast Enhancement Agents” filed Nov. 9, 2009 and having a docket number 233560-2 and Ser. No. 12/614,729.

### BACKGROUND

This invention relates to contrast enhancement agents for use in magnetic resonance imaging, more particularly to metal chelating ligands and metal chelate compounds useful in the preparation of such contrast enhancement agents.

Magnetic resonance (MR) imaging has become a critical medical diagnostic tool in human health. The use of MR contrast enhancement agents in MR imaging protocols has proven to be a valuable addition to the technique by improving both the quality of images obtained in an MR imaging procedure and the efficiency with which such images can be gathered. Known MR contrast enhancement agents suffer from a variety of deficiencies. For example, MR contrast enhancement agents containing gadolinium (Gd) chelates, while themselves are not toxic comprise gadolinium ion which in free ionic form is toxic. Contrast enhancement agents comprising chelates of manganese (Mn) may be subject to dissociation of the chelating ligand from the manganese metal center which is undesirable. Various other metal chelates may serve as MR contrast enhancement agents but are frequently less effective than gadolinium chelates and/or are not cleared from the body of the subject at sufficiently high rates following the imaging procedure.

Considerable effort and ingenuity has been expended to reduce the latent toxicity and control bio-distribution of MR contrast enhancement agents comprising gadolinium chelates. Potential MR contrast enhancement agents should exhibit good in-vivo and in-vitro stability, as well as prompt clearance from the body following an MR imaging procedure. MR contrast enhancement agents comprising a paramagnetic iron center are attractive because iron has an extensive and largely innocuous natural biochemistry as compared to gadolinium. This has led to increased interest in the use of iron-based materials as contrast agents for MR imaging.

There exists a need for additional iron-containing contrast enhancement agents for MR imaging that exhibit performance superior to or equivalent to known contrast enhancement agents while providing one or more additional advantages, such as improved image quality at lower patient dosages, greater patient tolerance and safety when higher doses are required, and improved clearance from the patient following the imaging procedure.

### BRIEF DESCRIPTION

In one embodiment, the present invention provides a protected ligand precursor having structure XX

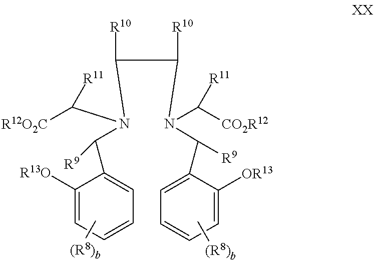

wherein R⁸ is independently at each occurrence a protected hydroxy group, a protected C₁-C₃ hydroxyalkyl group, or a C₁-C₃ alkyl group, and b is 0-4; R⁹-R¹¹ are independently at each occurrence hydrogen, a protected C₁-C₃ hydroxyalkyl group, or a C₁-C₃ alkyl group, with the proviso that at least one of R⁸-R¹¹ is a protected hydroxy group or a protected C₁-C₃ hydroxyalkyl group; and R¹² and R¹³ are independently at each occurrence a protecting group selected from the group consisting of C₁-C₃₀ aliphatic radicals, C₃-C₃₀ cycloaliphatic radicals, and C₂-C₃₀ aromatic radicals.

In another embodiment, the present invention provides a protected ligand precursor having structure XXIV

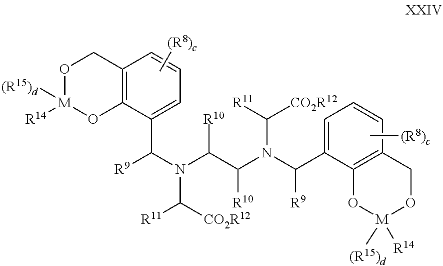

wherein R⁸ is independently at each occurrence a protected hydroxy group, a protected C₁-C₃ hydroxyalkyl group, or a C₁-C₃ alkyl group; R⁹-R¹¹ are independently at each occurrence hydrogen, a protected C₁-C₃ hydroxyalkyl group, or a C₁-C₃ alkyl group; R¹² is independently at each occurrence a protecting group selected from the group consisting of C₁-C₃₀ aliphatic radicals, C₃-C₃₀ cycloaliphatic radicals, and C₂-C₃₀ aromatic radicals; R¹⁴ and R¹⁵ are independently at each occurrence a C₁-C₁₀ alkyl group, a C₁-C₁₀ alkoxy group, or aryl group; M is independently at each occurrence B, Si or carbon; c is 0-3, and d is 0 or 1.

### DETAILED DESCRIPTION

In the following specification and the claims, which follow, reference will be made to a number of terms, which shall be defined to have the following meanings.

The singular forms “a”, “an” and “the” include plural referents unless the context clearly dictates otherwise.

“Optional” or “optionally” means that the subsequently described event or circumstance may or may not occur, and that the description includes instances where the event occurs and instances where it does not.

As used herein, the term “solvent” can refer to a single solvent or a mixture of solvents.

Approximating language, as used herein throughout the specification and claims, may be applied to modify any quantitative representation that could permissibly vary without resulting in a change in the basic function to which it is related. Accordingly, a value modified by a term or terms, such as “about”, is not to be limited to the precise value specified. In some instances, the approximating language may correspond to the precision of an instrument for measuring the value.

As used herein, the term “aromatic radical” refers to an array of atoms having a valence of at least one comprising at least one aromatic group. The array of atoms having a valence of at least one comprising at least one aromatic group may include heteroatoms such as nitrogen, sulfur, selenium, silicon and oxygen, or may be composed exclusively of carbon and hydrogen. As used herein, the term “aromatic radical” includes but is not limited to phenyl, pyridyl, furanyl, thienyl, naphthyl, phenylene, and biphenyl radicals. As noted, the aromatic radical contains at least one aromatic group. The aromatic group is invariably a cyclic structure having 4n+2 “delocalized” electrons where “n” is an integer equal to 1 or greater, as illustrated by phenyl groups (n=1), thienyl groups (n=1), furanyl groups (n=1), naphthyl groups (n=2), azulenyl groups (n=2), anthraceneyl groups (n=3) and the like. The aromatic radical may also include nonaromatic components. For example, a benzyl group is an aromatic radical which comprises a phenyl ring (the aromatic group) and a methylene group (the nonaromatic component). Similarly a tetrahydronaphthyl radical is an aromatic radical comprising an aromatic group (C₆H₃) fused to a nonaromatic component —(CH₂)₄—. For convenience, the term “aromatic radical” is defined herein to encompass a wide range of functional groups such as alkyl groups, alkenyl groups, alkynyl groups, haloalkyl groups, haloaromatic groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methylphenyl radical is a C₇ aromatic radical comprising a methyl group, the methyl group being a functional group which is an alkyl group. Similarly, the 2-nitrophenyl group is a C₆ aromatic radical comprising a nitro group, the nitro group being a functional group. Aromatic radicals include halogenated aromatic radicals such as 4-trifluoromethylphenyl, hexafluoro isopropylidenebis(4-phen-1-yloxy) (i.e., —OPhC(CF₃)₂PhO—), 4-chloromethylphen-1-yl, 3-trifluorovinyl-2-thienyl, 3-trichloromethylphen-1-yl (i.e., 3-CCl₃Ph-), 4-(3-bromoprop-1-yl)phen-1-yl (i.e., 4-BrCH₂CH₂CH₂Ph-), and the like. Further examples of aromatic radicals include 4-allyloxyphen-1-oxy, 4-aminophen-1-yl (i.e., 4-H₂NPh-), 3-aminocarbonylphen-1-yl (i.e., NH₂COPh-), 4-benzoylphen-1-yl, dicyanomethylidenebis(4-phen-1-yloxy) (i.e., —OPhC(CN)₂PhO—), 3-methylphen-1-yl, methylenebis(4-phen-1-yloxy) (i.e., —OPhCH₂PhO—), 2-ethylphen-1-yl, phenylethenyl, 3-formyl-2-thienyl, 2-hexyl-5-furanyl, hexamethylene-1,6-bis(4-phen-1-yloxy) (i.e., —OPh(CH₂)₆PhO—), 4-hydroxymethylphen-1-yl (i.e., 4-HOCH₂Ph-), 4-mercaptomethylphen-1-yl (i.e., 4-HSCH₂Ph-), 4-methylthiophen-1-yl (i.e., 4-CH₃SPh-), 3-methoxyphen-1-yl, 2-methoxycarbonylphen-1-yloxy (e.g., methyl salicyl), 2-nitromethylphen-1-yl (i.e., 2-NO₂CH₂Ph), 3-trimethylsilylphen-1-yl, 4-t-butyldimethylsilylphen-1-yl, 4-vinylphen-1-yl, vinylidenebis(phenyl), and the like. The term “a C₃-C₁₀ aromatic radical” includes aromatic radicals containing at least three but no more than 10 carbon atoms. The aromatic radical 1-imidazolyl (C₃H₂N₂—) represents a C₃ aromatic radical. The benzyl radical (C₇H₇—) represents a C₇ aromatic radical.

As used herein the term “cycloaliphatic radical” refers to a radical having a valence of at least one, and comprising an array of atoms which is cyclic but which is not aromatic. As defined herein a “cycloaliphatic radical” does not contain an aromatic group. A “cycloaliphatic radical” may comprise one or more noncyclic components. For example, a cyclohexylmethyl group (C₆H₁₁CH₂—) is a cycloaliphatic radical which comprises a cyclohexyl ring (the array of atoms which is cyclic but which is not aromatic) and a methylene group (the noncyclic component). The cycloaliphatic radical may include heteroatoms such as nitrogen, sulfur, selenium, silicon and oxygen, or may be composed exclusively of carbon and hydrogen. For convenience, the term “cycloaliphatic radical” is defined herein to encompass a wide range of functional groups such as alkyl groups, alkenyl groups, alkynyl groups, haloalkyl groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methylcyclopent-1-yl radical is a C₆ cycloaliphatic radical comprising a methyl group, the methyl group being a functional group which is an alkyl group. Similarly, the 2-nitrocyclobut-1-yl radical is a C₄ cycloaliphatic radical comprising a nitro group, the nitro group being a functional group. A cycloaliphatic radical may comprise one or more halogen atoms which may be the same or different. Halogen atoms include, for example; fluorine, chlorine, bromine, and iodine. Cycloaliphatic radicals comprising one or more halogen atoms include 2-trifluoromethylcyclohex-1-yl, 4-bromodifluoromethylcyclooct-1-yl, 2-chlorodifluoromethylcyclohex-1-yl, hexafluoroisopropylidene-2,2-bis(cyclohex-4-yl) (i.e., —C₆H₁₀C(CF₃)₂C₆H₁₀—), 2-chloromethylcyclohex-1-yl, 3-difluoromethylenecyclohex-1-yl, 4-trichloromethylcyclohex-1-yloxy, 4-bromodichloromethylcyclohex-1-ylthio, 2-bromoethylcyclopent-1-yl, 2-bromopropylcyclohex-1-yloxy (e.g., CH₃CHBrCH₂C₆H₁₀O—), and the like. Further examples of cycloaliphatic radicals include 4-allyloxycyclohex-1-yl, 4-aminocyclohex-1-yl (i.e., H₂C₆H₁₀—), 4-aminocarbonylcyclopent-1-yl (i.e., NH₂COC₅H₈—), 4-acetyloxycyclohex-1-yl, 2,2-dicyanoisopropylidenebis(cyclohex-4-yloxy) (i.e., —OC₆H₁₀C(CN)₂C₆H₁₀O—), 3-methylcyclohex-1-yl, methylenebis(cyclohex-4-yloxy) (i.e., —OC₆H₁₀CH₂C₆H₁₀O—), 1-ethylcyclobut-1-yl, cyclopropylethenyl, 3-formyl-2-terahydrofuranyl, 2-hexyl-5-tetrahydrofuranyl, hexamethylene-1,6-bis(cyclohex-4-yloxy) (i.e., —OC₆H₁₀(CH₂)₆C₆H₁₀O—), 4-hydroxymethylcyclohex-1-yl (i.e., 4-HOCH₂C₆H₁₀—), 4-mercaptomethylcyclohex-1-yl (i.e., 4-HSCH₂C₆H₁₀—), 4-methylthiocyclohex-1-yl (i.e., 4-CH₃SC₆H₁₀—), 4-methoxycyclohex-1-yl, 2-methoxycarbonylcyclohex-1-yloxy (2-CH₃OCOC₆H₁₀O—), 4-nitromethylcyclohex-1-yl (i.e., NO₂CH₂C₆H₁₀—), 3-trimethylsilylcyclohex-1-yl, 2-t-butyldimethylsilylcyclopent-1-yl, 4-trimethoxysilylethylcyclohex-1-yl (e.g., (CH₃O)₃SiCH₂CH₂C₆H₁₀—), 4-vinylcyclohexen-1-yl, vinylidenebis(cyclohexyl), and the like. The term “a C₃-C₁₀ cycloaliphatic radical” includes cycloaliphatic radicals containing at least three but no more than 10 carbon atoms. The cycloaliphatic radical 2-tetrahydrofuranyl (C₄H₇O—) represents a C₄ cycloaliphatic radical. The cyclohexylmethyl radical (C₆H₁₁CH₂—) represents a C₇ cycloaliphatic radical.

As used herein the term “aliphatic radical” refers to an organic radical having a valence of at least one consisting of a linear or branched array of atoms which is not cyclic. Aliphatic radicals are defined to comprise at least one carbon atom. The array of atoms comprising the aliphatic radical may include heteroatoms such as nitrogen, sulfur, silicon, selenium and oxygen or may be composed exclusively of carbon and hydrogen. For convenience, the term “aliphatic radical” is defined herein to encompass, as part of the “linear or branched array of atoms which is not cyclic” a wide range of functional groups such as alkyl groups, alkenyl groups, alkynyl groups, haloalkyl groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methylpent-1-yl radical is a C₆ aliphatic radical comprising a methyl group, the methyl group being a functional group which is an alkyl group. Similarly, the 4-nitrobut-1-yl group is a C₄ aliphatic radical comprising a nitro group, the nitro group being a functional group. An aliphatic radical may be a haloalkyl group which comprises one or more halogen atoms which may be the same or different. Halogen atoms include, for example; fluorine, chlorine, bromine, and iodine. Aliphatic radicals comprising one or more halogen atoms include the alkyl halides trifluoromethyl, bromodifluoromethyl, chlorodifluoromethyl, hexafluoroisopropylidene, chloromethyl, difluorovinylidene, trichloromethyl, bromodichloromethyl, bromoethyl, 2-bromotrimethylene (e.g., —CH₂CHBrCH₂—), and the like. Further examples of aliphatic radicals include allyl, aminocarbonyl (i.e., —CONH₂), carbonyl, 2,2-dicyanoisopropylidene (i.e., —CH₂C(CN)₂CH₂—), methyl (i.e., —CH₃), methylene (i.e., —CH₂—), ethyl, ethylene, formyl (i.e., —CHO), hexyl, hexamethylene, hydroxymethyl (i.e., —CH₂OH), mercaptomethyl (i.e., —CH₂SH), methylthio (i.e., —SCH₃), methylthiomethyl (i.e., —CH₂SCH₃), methoxy, methoxycarbonyl (i.e., CH₃OCO—), nitromethyl (i.e., —CH₂NO₂), thiocarbonyl, trimethylsilyl (i.e., (CH₃)₃Si—), t-butyldimethylsilyl, 3-trimethyoxysilylpropyl (i.e., (CH₃O)₃SiCH₂CH₂CH₂—), vinyl, vinylidene, and the like. By way of further example, a C₁-C₁₀ aliphatic radical contains at least one but no more than 10 carbon atoms. A methyl group (i.e., CH₃—) is an example of a C₁ aliphatic radical. A decyl group (i.e., CH₃(CH₂)₉—) is an example of a C₁₀ aliphatic radical.

As noted, in one embodiment, the present invention provides a contrast enhancement agent comprising an iron chelate having structure I

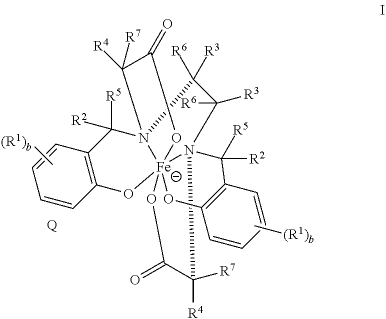

wherein R¹ is independently at each occurrence a hydroxy group, a C₁-C₃ hydroxyalkyl group, or a C₁-C₃ alkyl group, and b is 0-4; R²-R⁷ are independently at each occurrence hydrogen, a C₁-C₃ hydroxyalkyl group, or a C₁-C₃ alkyl group, with the proviso that at least one of R¹-R⁷ is a hydroxy group or a C₁-C₃ hydroxyalkyl group; and wherein Q is a charge balancing counterion.

Although throughout this disclosure there is considerable focus on human health, the contrast enhancement agents provided by the present invention are useful in the study and treatment of variety of human and animal diseases as imaging agents, and as probes for the development of imaging agents.

Contrast enhancement agents comprising an iron chelate and falling within generic structure I are illustrated in Table 1 below

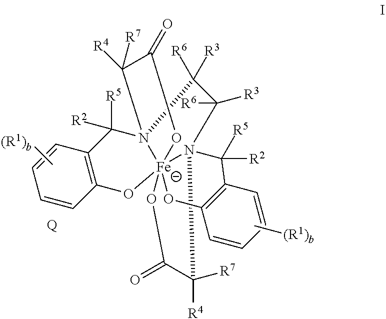

TABLE 1
Examples of Iron Chelate Contrast Enhancement Agents Having Structure I

| | | | Variable |
|---|---|---|---|
| | | Variables R¹—R⁷ | Q Defined |
| Entry | Structure | Defined As | As |
| 1a | 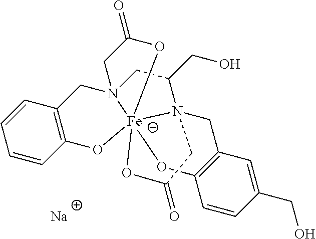 | R¹ is hydroxymethyl; R²—R⁵ are hydrogen; R⁶ is hydroxymethyl and hydrogen; R⁷ is hydrogen; b is 0 and 1. | Na⁺ |
| 1b | 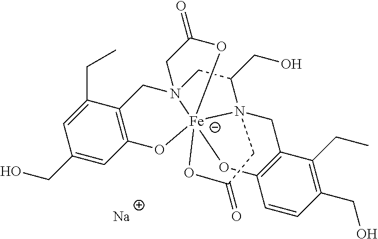 | R¹ is hydroxymethyl and ethyl; R²—R⁵ are hydrogen; R⁶ is hydroxymethyl and hydrogen; R⁷ is hydrogen; b is 2. | Na⁺ |
| 1c | 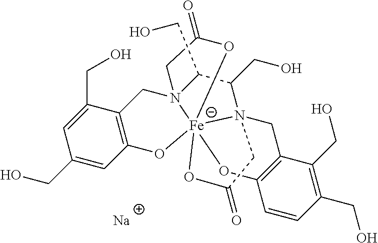 | R¹ is hydroxymethyl; R²—R⁵ are hydrogen, R⁶ is hydroxymethyl; R⁷ is hydrogen; b is 2. | Na⁺ |
| 1d | 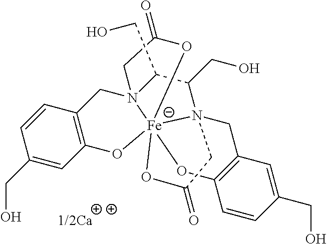 | R¹ is hydroxymethyl; R²—R⁵ are hydrogen; R⁶ is hydroxymethyl; R⁷ is hydrogen; b is 1. | ½ Ca⁺⁺ |
| 1e | 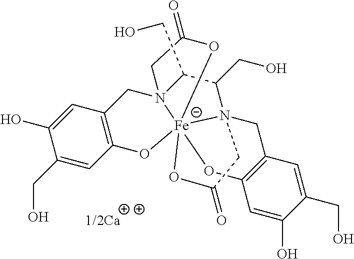 | R¹ is hydroxy and hydroxymethyl; R²—R⁵ are hydrogen; R⁶ is hydroxymethyl; R⁷ is hydrogen; b is 2. | ½ Ca⁺⁺ |

In general, and throughout this disclosure, no absolute or relative stereochemistry is intended to be shown for a structure, as in for example structures I and II, and the structures are intended to encompass all possible absolute and relative stereochemical configurations, unless specified otherwise. Thus, structure I depicts an iron chelate compound in which no absolute or relative stereochemistry is intended to be shown. As such, structure I is intended to represent a genus of iron chelate compounds which includes racemic compounds, single enantiomers, enantiomerically enriched compositions and mixtures of diastereomers. In one embodiment, the present invention provides a contrast enhancement agent having structure 1a (Table 1) which is a racemic mixture having equal concentrations of levorotatory and dextrorotatory enantiomers of contrast enhancement agent 1a. In an alternate embodiment, the present invention provides a contrast enhancement agent having structure 1b (Table 1) which is an enantiomerically enriched mixture having unequal concentrations of levorotatory and dextrorotatory enantiomers of 1b. In yet another embodiment, the present invention provides a contrast enhancement agent having structure 1c (Table 1) which is a diastereomeric mixture comprising at least two compounds having structure 1c which are not enantiomers.

Those skilled in the art will appreciate that the iron chelate compositions provided by the present invention may comprise a principal component enantiomer, a minor component enantiomer, and additional diastereomeric iron chelate components. In one embodiment, the present invention provides an iron chelate composition comprising a principal component enantiomer and related diastereomers. In an alternate embodiment, the present invention provides an iron chelate composition having no principal component enantiomer and which is a diastereomeric mixture.

In another embodiment, the present invention provides a contrast enhancement agent comprising an iron chelate having structure II

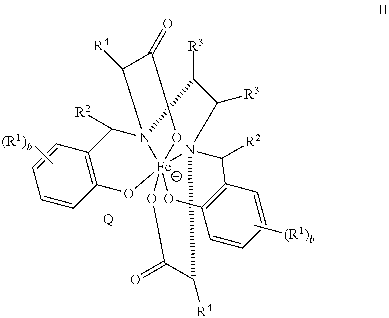

wherein R¹ is independently at each occurrence a hydroxy group, a C₁-C₃ hydroxyalkyl group, or a C₁-C₃ alkyl group, and b is 0-4; R²-R⁴ are hydrogen, a C₁-C₃ hydroxyalkyl group, or a C₁-C₃ alkyl group, with the proviso that at least one of R¹-R⁴ is a hydroxy group or a C₁-C₃ hydroxyalkyl group; and wherein Q is a charge balancing counterion.

Contrast enhancement agents comprising an iron chelate and falling within generic structure II are illustrated in Table 2 below.

TABLE 2
Examples of Iron Chelate Contrast Enhancement Agents Having Structure II

| | | Variables R¹—R⁴ | Variable Q |
|---|---|---|---|
| Entry | Structure | Defined As | Defined As |
| 2a | 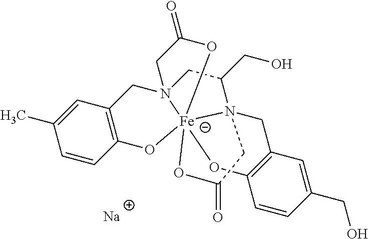 | R¹ is methyl and hydroxymethyl; R² and R⁴ are hydrogen; R³ is hydroxymethyl and hydrogen; b is 1. | Na⁺ |
| 2b | 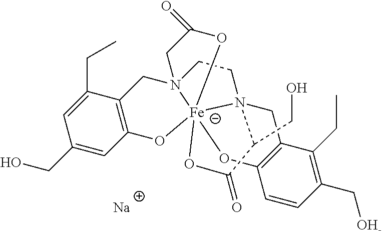 | R¹ is hydroxymethyl and ethyl; R²—R³ are hydrogen; R⁴ is hydroxymethyl and hydrogen; b is 2. | Na⁺ |
| 2c | 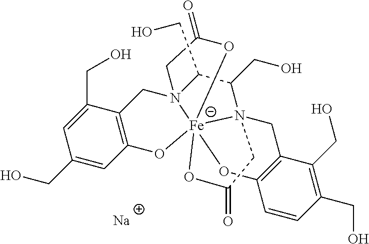 | R¹ is hydroxymethyl; R² and R⁴ are hydrogen; R³ is hydroxymethyl; b is 2. | Na⁺ |
| 2d | 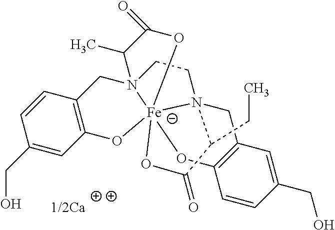 | R¹ is hydroxymethyl; R²— R³ are hydrogen; R⁴ is methyl and ethyl; b is 1. | ½ Ca⁺⁺ |
| 2e | 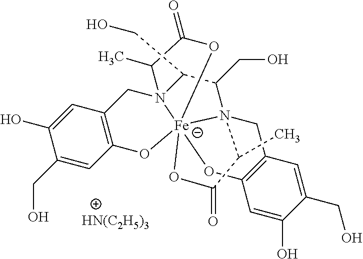 | R¹ is hydroxy and hydroxymethyl; R² is hydrogen; R³ is hydroxymethyl; R⁴ is methyl; b is 2. | ⁺HN(C₂H₅)₃ |

The charge balancing counterion Q may be an organic cation or an inorganic cation. Thus, in one embodiment, the charge balancing counterion Q is an inorganic cation. Non-limiting examples of inorganic cations include alkali metal cations, alkaline earth metal cations, transition metal cations, and inorganic ammonium cations (NH₄⁺). In another embodiment, the charge balancing counterion Q is an organic cation, for example an organic ammonium cation, an organic phosphonium cation, an organic sulfonium cation, or a mixture thereof. In one embodiment, the charge balancing counterion is the ammonium salt of an aminosugar such as the 2-(N,N,N-trimethylammonium)-2-deoxyglucose. In one embodiment, the charge balancing counterion is the protonated form of N-methyl glucamine.

In one embodiment, the contrast enhancing agent includes an iron chelate having structure III

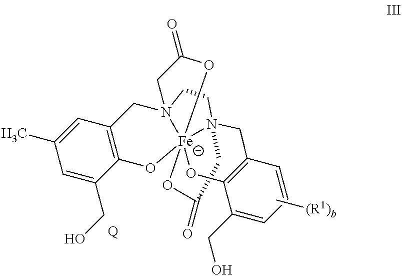

wherein Q is a charge balancing counterion.

In another embodiment, the contrast enhancing agent includes an iron chelate having structure IV

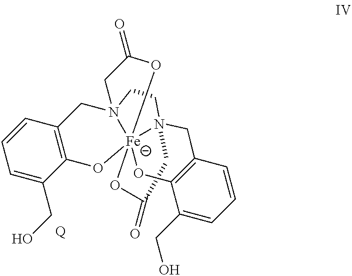

wherein Q is a charge balancing counterion.

In another embodiment, the contrast enhancing agent includes an iron chelate having structure V

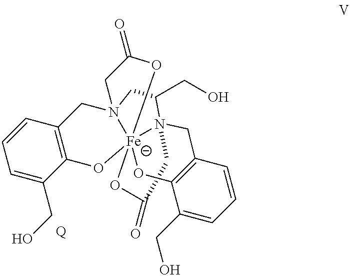

wherein Q is a charge balancing counterion.

In yet another embodiment, the contrast enhancing agent includes an iron chelate having structure VI

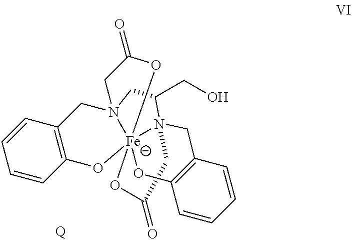

wherein Q is a charge balancing counterion.

In another embodiment, the contrast enhancing agent includes an iron chelate having structure VII

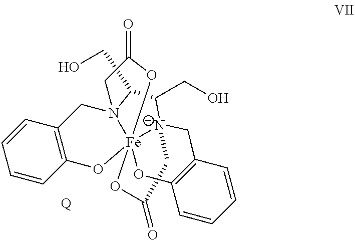

wherein Q is a charge balancing counterion. In yet another embodiment, the contrast enhancing agent includes an iron chelate having structure VIII

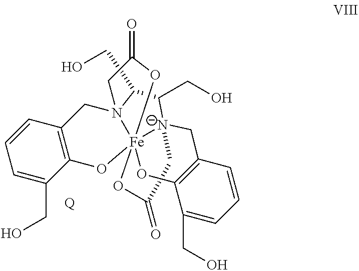

wherein Q is a charge balancing counterion.

In one embodiment, the present invention provides a metal chelating ligand having idealized structure IX

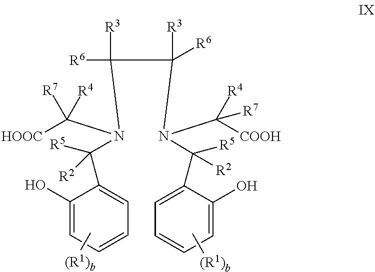

wherein R¹ is independently at each occurrence a hydroxy group, a C₁-C₃ hydroxyalkyl group, or a C₁-C₃ alkyl group, and b is 0-4; R²-R⁷ are independently at each occurrence hydrogen, a C₁-C₃ hydroxyalkyl group, or a C₁-C₃ alkyl group, with the proviso that at least one of R¹-R⁷ is a hydroxy group or a C₁-C₃ hydroxyalkyl group.

The term “idealized structure” is used herein to designate the structure indicated and additional structures which may include protonated and deprotonated forms of the metal chelating ligand having the idealized structure. Those having ordinary skill in the art will appreciate that the individual metal chelating ligands provided by the present invention may comprise protonated and deprotonated forms of the metal chelating ligand, for example the idealized structure of metal chelating ligand of structure IX comprises one or more of the protonated and the deprotonated forms having structure X-XII

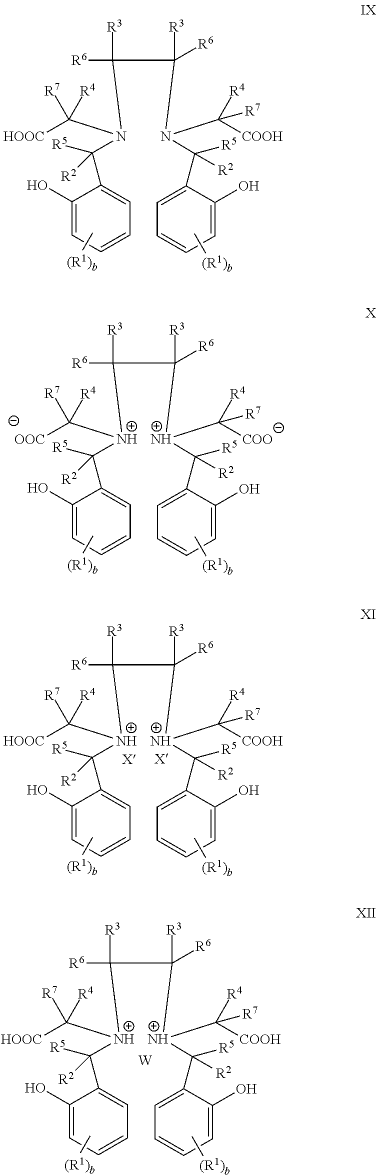

wherein W and X′ are charge balancing counterions. In one embodiment, the charge balancing counterion X′ may be an inorganic anion or an organic anion. Similarly, W may be an inorganic anion or an organic anion. Thus, in one embodiment, the charge balancing counterion W is an inorganic anion. In another embodiment, the charge balancing counterion W is an organic anion. Similarly, in one embodiment, the charge balancing counterion X′ is an inorganic anion. In another embodiment, the charge balancing counterion X′ is an organic anion. Those skilled in the art will appreciate that charge balancing counterions X′ include monovalent anions such as chloride, bromide, iodide, bicarbonate, acetate, glycinate, ammonium succinate, and the like. Similarly, those skilled in the art will appreciate that charge balancing counterions W include polyvalent anions such as carbonate, sulfate, succinate, malonate, and the like.

Metal chelating ligands having idealized structure IX are further illustrated in Table 3 below.

TABLE 3
Examples of Metal Chelating Ligands Having Idealized Structure IX

| | | Variables R¹-R⁷ | | |
|---|---|---|---|---|
| Entry | Structure | Defined As | W | X′ |
| 3a | 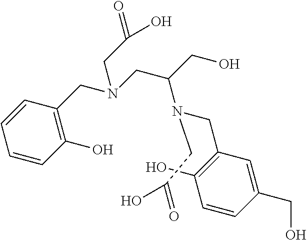 | R¹ is hydroxymethyl; R²—R⁵ are hydrogen; R⁶ is hydroxymethyl and hydrogen; R⁷ is hydrogen; b is 0 and 1. | — | — |
| 3b | 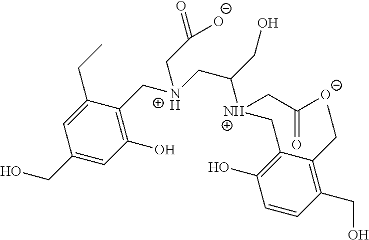 | R¹ is hydroxymethyl and ethyl; R²—R⁵ are hydrogen; R⁶ is hydroxymethyl and hydrogen; R⁷ is hydrogen; b is 2. | — | — |
| 3c | 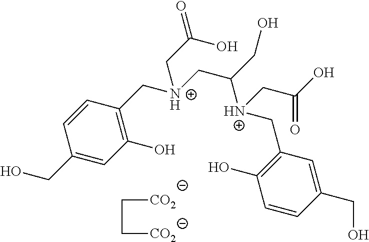 | R¹ is hydroxymethyl; R²—R⁵ are hydrogen; R⁶ is hydroxymethyl and hydrogen; R⁷ is hydrogen; b is 1. | 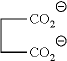 (succinate) | — |
| 3d | 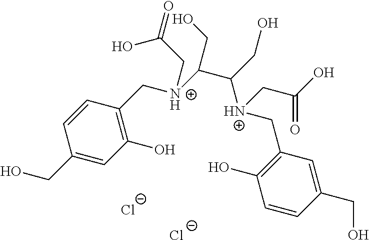 | R¹ is hydroxymethyl; R²—R⁵ are hydrogen; R⁶ is hydroxymethyl; R⁷ is hydrogen; b is 1. | — | Cl⁻ |

In an alternate embodiment, the present invention provides a metal chelating ligand having an idealized structure XIII

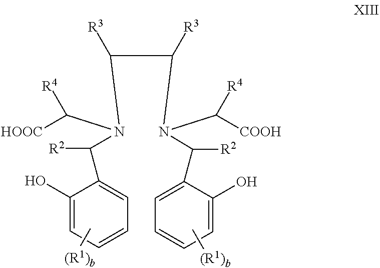

wherein R¹ is independently at each occurrence a hydroxy group, a C₁-C₃ hydroxyalkyl group, or a C₁-C₃ alkyl group, and b is 0-4; and R²-R⁴ are hydrogen, a C₁-C₃ hydroxyalkyl group, or a C₁-C₃ alkyl group, with the proviso that at least one of R¹-R⁴ is a hydroxy group or C₁-C₃ hydroxyalkyl group.

The metal chelating ligands having idealized structure XIII are illustrated in Table 4 below.

TABLE 4
Examples of Metal Chelating Ligands Having Idealized Structure XIII

| | | Variables R¹—R⁴ | | |
|---|---|---|---|---|
| Entry | Structure | Defined As | W | X′ |
| 4a | 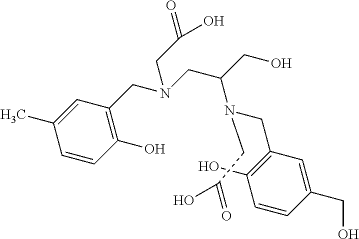 | R¹ is methyl and hydroxymethyl; R² and R⁴ are hydrogen; R³ is hydroxymethyl and hydrogen; b is 1. | — | — |
| 4b | 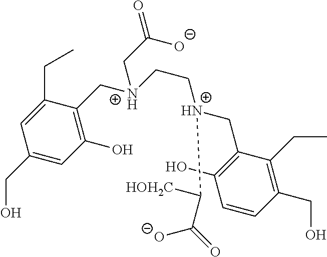 | R¹ is hydroxymethyl and ethyl; R²—R³ are hydrogen; R⁴ is hydroxymethyl and hydrogen; b is 2. | — | — |
| 4c | 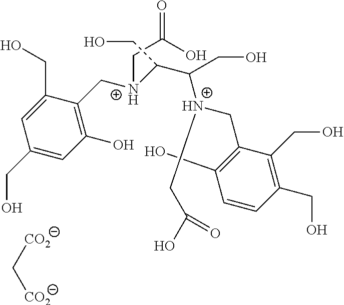 | R¹ is hydroxymethyl; R² and R⁴ are hydrogen; R³ is hydroxymethyl; b is 2. | 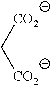 (malonate) | — |
| 4d | 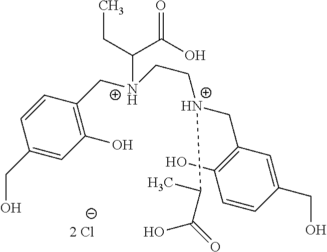 | R¹ is hydroxymethyl; R²—R³ are hydrogen; R⁴ is methyl and ethyl; b is 1. | — | Cl⁻ |
| 4e | 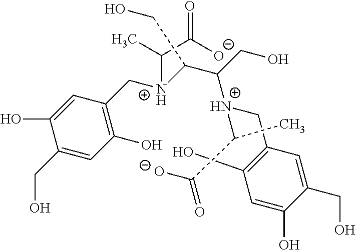 | R¹ is hydroxy and hydroxymethyl; R² is hydrogen R³ is hydroxymethyl; R⁴ is methyl; b is 2. | — | — |

The metal chelating ligands form coordinate complexes with a variety of metals. In one embodiment, the metal chelating ligands form complexes with transition metals. In a particular embodiment, the transition metal is iron.

In one embodiment, the metal chelating ligand has an idealized structure XIV. The preparation of a composition having idealized structure XIV is given in Example 5 of the Examples section of this disclosure.

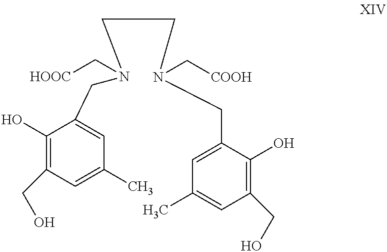

In another embodiment, the metal chelating ligand has an idealized structure XV. The preparation of a composition having idealized structure XV is given in Example 2 of the Examples section of this disclosure.

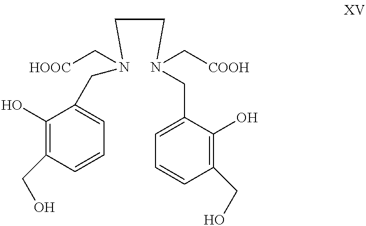

In yet another embodiment, the metal chelating ligand has an idealized structure XVI.

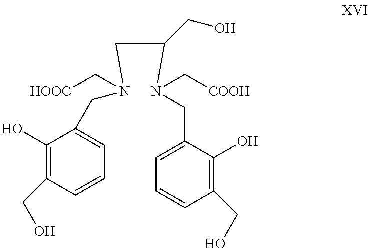

In another embodiment, the metal chelating ligand has an idealized structure XVII.

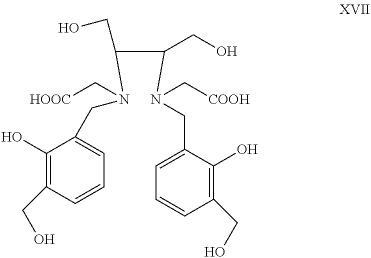

In one embodiment, the present invention provides a partially deprotected ligand precursor XVIII having free carboxylic acid groups (or ionized forms thereof)

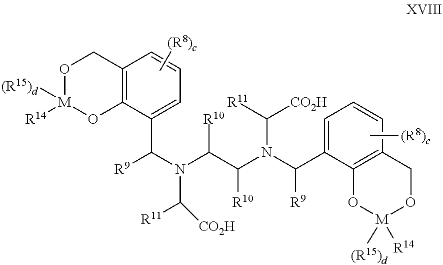

wherein with respect only to structure XVIII, R⁸ is independently at each occurrence a hydroxy group, a protected hydroxy group, a C₁-C₃ hydroxyalkyl group, a protected C₁-C₃ hydroxyalkyl group, or a C₁-C₃ alkyl group; R⁹-R¹¹ are independently at each occurrence hydrogen, a C₁-C₃ hydroxyalkyl group, a protected C₁-C₃ hydroxyalkyl group, or a C₁-C₃ alkyl group; R¹⁴ and R¹⁵ are independently at each occurrence a C₁-C₁₀ alkyl group, a C₁-C₁₀ alkoxy group, or an aryl group; M is independently at each occurrence a B, Si or carbon; c is 0-3; and d is 0 or 1. The ligand precursor XVIII may be converted to a metal chelating ligand as is demonstrated in the Examples section of this disclosure.

The partially protected ligand precursors falling within generic structure XVIII are illustrated in Table 5 below.

TABLE 5
Examples Partially Deprotected Ligand Precursors XVIII Having Structure XVIII

| | | Variables c, d, R⁸—R¹¹, R¹⁴, R¹⁵ |
|---|---|---|
| Entry | Structure | and M Defined As |
| 5a | 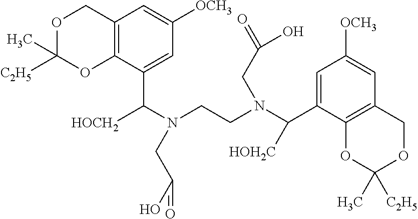 | R⁸ is OCH₃; R⁹ is CH₂OH, R¹⁰ and R¹¹ are hydrogen, c is 1; d is 1; M is carbon, R¹⁴ is methyl and R¹⁵ is ethyl. |
| 5b | 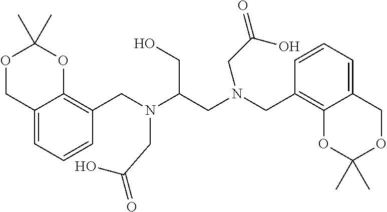 | R⁹ is hydrogen; R¹⁰ is hydroxymethyl and hydrogen; c is 0; d is 1; M is carbon, R¹⁴ and R¹⁵ are CH₃. |
| 5c | 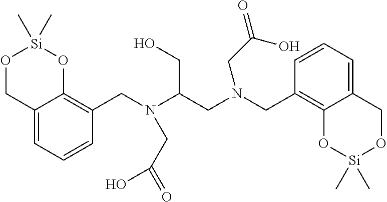 | R⁹ is hydrogen; R¹⁰ is hydroxymethyl and hydrogen; c is 0; d is 1; M is silicon (Si); and R¹⁴ and R¹⁵ are CH₃. |
| 5d | 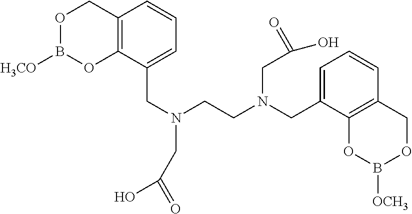 | R⁹—R¹⁰ are hydrogen; c is 0; d is 0; M is boron (B); and R¹⁴ is methoxy (OCH₃). |

In one embodiment, the present invention provides a partially deprotected ligand precursor falling within the generic structure XVIII having structure XIX.

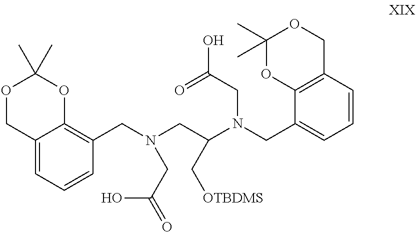

In one embodiment, the present invention provides a partially deprotected ligand precursor corresponding to XVIII wherein the group R¹⁵ is phenyl.

In one embodiment, the present invention provides protected ligand precursors that may be employed for the synthesis of the contrast enhancement agents. In one embodiment, the protected ligand precursor has a structure XX

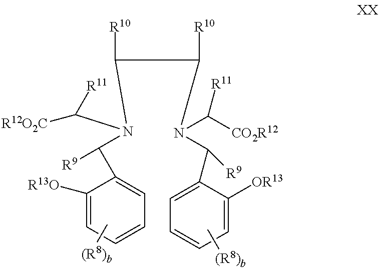

wherein R⁸ is independently at each occurrence a protected hydroxy group, a protected C₁-C₃ hydroxyalkyl group, or a C₁-C₃ alkyl group, and b is 0-4; R⁹-R¹¹ are independently at each occurrence hydrogen, a protected C₁-C₃ hydroxyalkyl group, or a C₁-C₃ alkyl group, with the proviso that at least one of R⁸-R¹¹ is a protected hydroxy group or a protected C₁-C₃ hydroxyalkyl group; and R¹² and R¹³ are independently at each occurrence a protecting group selected from the group consisting of C₁-C₃₀ aliphatic radicals, C₃-C₃₀ cycloaliphatic radicals, and C₂-C₃₀ aromatic radicals. A wide variety of protecting groups may be incorporated into the protected ligand precursors provided by the present invention. These include acid sensitive protecting groups (for example the methylthiomethyl group), base sensitive protecting groups for example the acetate and trichloroacetate groups), light sensitive protecting groups (for example the ortho-nitrobenzyl group), groups susceptible to hydrogenolysis (for example the benzyl group), and groups susceptible to metal mediated transformations which enhance their lability (for example the allyl group).

In one embodiment, the present invention provides a protected ligand precursor having structure XX wherein R¹² is independently at each occurrence an ethyl group, a trichloroethyl group, a beta-cyanoethyl group, a trimethylsilyl ethyl group, or a tertiary butyl group. In one embodiment, the present invention provides a protected ligand precursor having structure XX wherein R¹² is independently at each occurrence an ethyl group. In an alternate embodiment, the present invention provides a protected ligand precursor having structure XX wherein R¹² is independently at each occurrence a trichloroethyl group. In yet another embodiment, the present invention provides a protected ligand precursor having structure XX wherein R¹² is independently at each occurrence a beta-cyanoethyl group. In yet still another embodiment, the present invention provides a protected ligand precursor having structure XX wherein R¹² is independently at each occurrence a trimethylsilyl ethyl group. In yet another embodiment, the present invention provides a protected ligand precursor having structure XX wherein R¹² is independently at each occurrence a tertiary butyl group.

Protected ligand precursors falling within generic structure XX are illustrated in Table 6 below.

TABLE 6
Examples of Protected Ligands Precursor Having Structure XX

| | | Varables b and R⁸— |
|---|---|---|
| Entry | Structure | R¹³ Defined As |
| 6a | 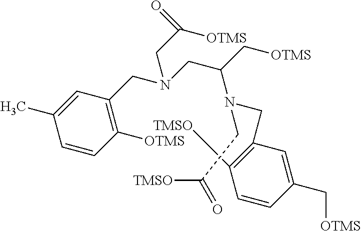 | R⁸ is methyl and protected hydroxymethyl (CH₂OTMS); R⁹ and R¹¹ are hydrogen; R¹⁰ is protected hydroxymethyl (CH₂OTMS) and hydrogen; b is 1; R¹² is trimethylsilyl; R¹³ is trimethylsilyl. |
| 6b | 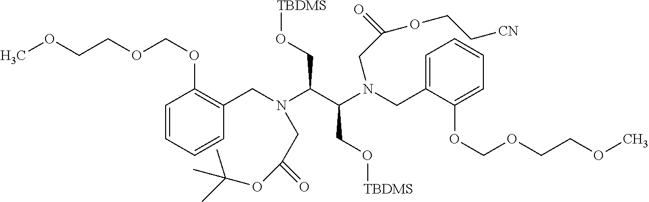 | R⁹ and R¹¹ are hydrogen; R¹⁰ is protected hydroxymethyl (CH₂OTBDMS); b is ( ); R¹² is t-butyl and beta-cyanoethyl; R¹³ is CH₃OCH₂CH₂OCH₂. |
| 6c | 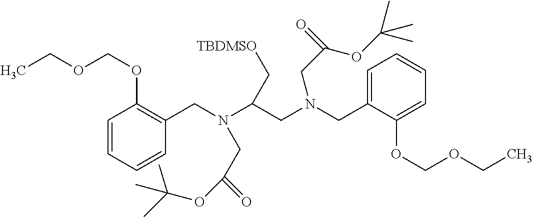 | R⁹ and R¹¹ are hydrogen; R¹⁰ is protected hydroxymethyl (CH₂OTBDMS); b is 0; R¹² is t-butyl; R¹³ is C₂H₅OCH₂. |
| 6d | 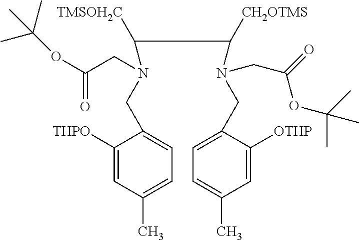 | R⁸ is methyl; R⁹ and R¹¹ are hydrogen; R¹⁰ is protected hydroxymethyl (CH₂OTMS); b is 1; R¹² is t-butyl; R¹³ is THP (tetrahydropyranyl). |

In one embodiment, the present invention provides protected ligand precursor having structure XX wherein R¹² and R¹³ are independently at each occurrence an acid sensitive protecting group. Non-limiting examples of acid sensitive protecting groups include an acetal group, a ketal group, a methoxthyethoxymethyl group, a t-butyl group, a t-butyldimethylsilyl group, a trimethylsilyl group, and a trimethylsilyl ethyl group. In one embodiment, R¹² is a tertiary butyl group. In another embodiment, R¹² is a trimethylsilyl group. In another embodiment, R¹² is a tert-butyldimethylsilyl group. In yet another embodiment, R¹² is a trimethylsilyl ethyl group. In one embodiment, R¹³ is a THP group. In another embodiment, R¹³ is a methoxthyethoxymethyl group. In another embodiment, R¹³ is a t-butyldimethylsilyl group. In yet another embodiment, R¹³ is a trimethylsilyl group.

In one embodiment, the present invention provides a protected ligand precursor having structure XXI.

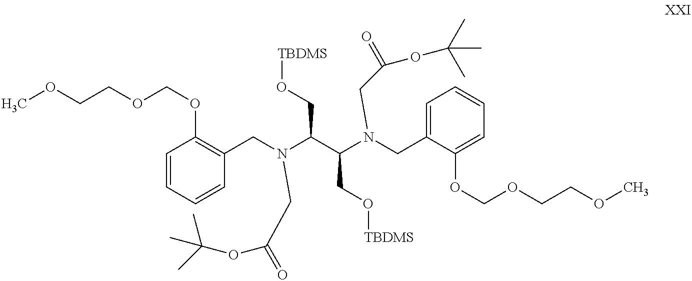

In another embodiment, the present invention provides a protected ligand precursor having structure XXII.

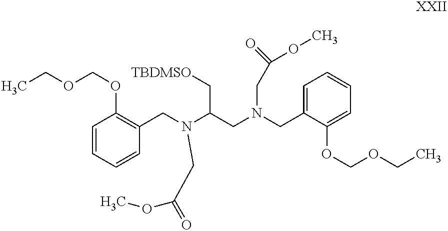

In one embodiment, the present invention provides a protected ligand precursor having structure XXIII

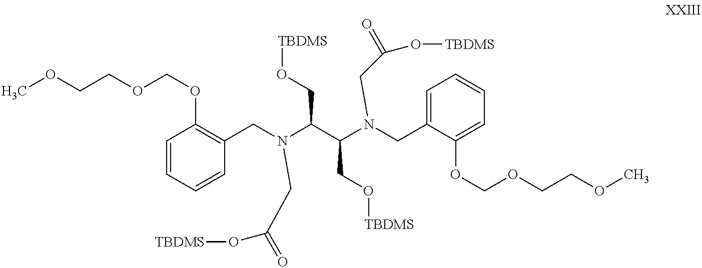

In one embodiment, the present invention provides a protected ligand precursor having structure XXIV

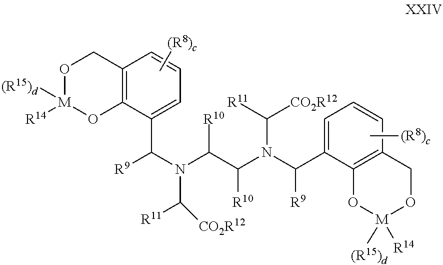

wherein R⁸ is independently at each occurrence a protected hydroxy group, a protected C₁-C₃ hydroxyalkyl group, or a C₁-C₃ alkyl group; R⁹-R¹¹ are independently at each occurrence hydrogen, a protected C₁-C₃ hydroxyalkyl group, or a C₁-C₃ alkyl group; R¹² is independently at each occurrence a protecting group selected from the group consisting of C₁-C₃₀ aliphatic radicals, C₃-C₃₀ cycloaliphatic radicals, and C₂-C₃₀ aromatic radicals; R¹⁴ and R¹⁵ are independently at each occurrence hydrogen, a C₁-C₁₀ alkyl group, a C₁-C₁₀ alkoxy group, or an aryl group; or the groups R¹⁴ and R¹⁵ may together with M form a carbonyl group or a thiocarbonyl group; M is independently at each occurrence B, Si or carbon; c is 0-3; and d is 0 or 1.

In one embodiment, the present invention provides a protected ligand precursor having structure XXIV wherein R¹² is independently at each occurrence an ethyl group, a trichloroethyl group, a beta-cyanoethyl group, a trimethylsilyl ethyl group, or a tertiary butyl group. In one embodiment, the present invention provides a protected ligand precursor having structure XXIV wherein R¹² is independently at each occurrence an ethyl group. In an alternate embodiment, the present invention provides a protected ligand precursor having structure XXIV wherein R¹² is independently at each occurrence a trichloroethyl group. In yet another embodiment, the present invention provides a protected ligand precursor having structure XXIV wherein R¹² is independently at each occurrence a beta-cyanoethyl group. In yet still another embodiment, the present invention provides a protected ligand precursor having structure XXIV wherein R¹² is independently at each occurrence a trimethylsilyl ethyl group. In yet another embodiment, the present invention provides a protected ligand precursor having structure XXIV wherein R¹² is independently at each occurrence a tertiary butyl group.

Protected ligand precursors falling within generic structure XXIV are illustrated in Table 7 below.

TABLE 7
Examples of Protected Ligand Precursors Having Structure XXIV

| | | Variables c, d, R⁸—R¹², R¹⁴, R¹⁵ |
|---|---|---|
| Entry | Structure | and M Defined As |
| 7a | 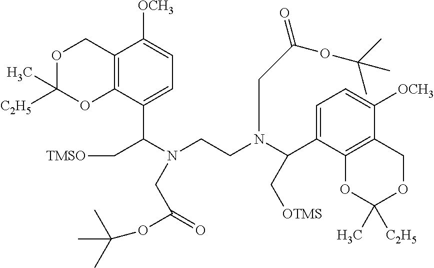 | R⁸ is OCH3; c is 1; d is 1; R⁹ is protected hydroxymethyl (CH₂OTMS); R¹⁰ and R¹¹ are hydrogen; R¹² is t-butyl; M is carbon, R¹⁴ is methyl group and R¹⁵ is ethyl. |
| 7b | 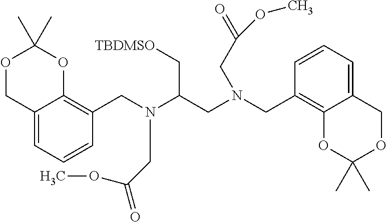 | R⁹ and R¹¹ are hydrogen; R¹⁰ is protected hydroxymethyl (CH₂OTBDMS); c is 0; d is 1; R¹² is methyl; R¹⁴ and R¹⁵ are CH₃ |
| 7c | 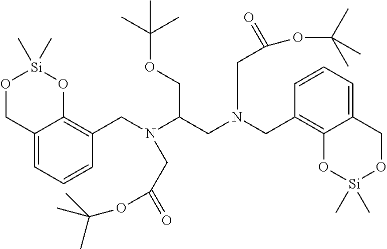 | R⁹ and R¹¹ are hydrogen; R¹⁰ is protected hydroxymethyl (CH₂O-t-butyl); c is 0; d is 1; R¹² is t-butyl; M is Si; and R¹⁴ and R¹⁵ are CH₃. |
| 7d | 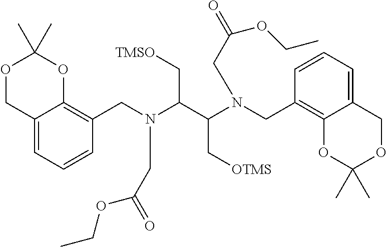 | R⁹ and R¹¹ are hydrogen; R¹⁰ is protected hydroxymethyl (CH₂OTMS); c is 0; d is 1; R¹² is ethyl; M is carbon, R¹⁴ and R¹⁵ are CH₃. |

In one embodiment, protected ligand precursor having structure XXIV the R¹² is independently at each occurrence an acid sensitive protecting group selected from the group consisting of an acetal group, a ketal group, methoxthyethoxymethyl group, t-butyl group, t-butyldimethylsilyl group, trimethylsilyl group, trimethylsilyl ethyl group. In one embodiment, the R¹² is a tertiary butyl group. In another embodiment, the R¹² is a trimethylsilyl group. In another embodiment, the R¹² is a tert-butyldimethylsilyl group. In yet another embodiment, the R¹² is a trimethylsilyl ethyl group.

In a particular embodiment, the present invention provides a protected ligand precursor corresponding to XXIV wherein the group R¹⁵ is phenyl, for example as in the case in which M carbon and R¹⁴ is methyl.

In one embodiment, the present invention provides a protected ligand precursor having structure XXV.

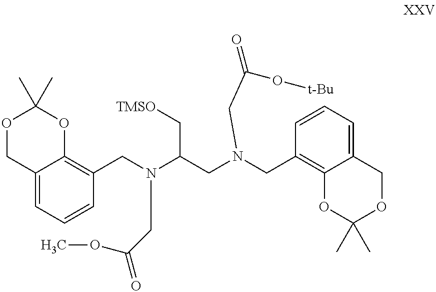

In another embodiment, the present invention provides a protected ligand precursor having structure XXVI.

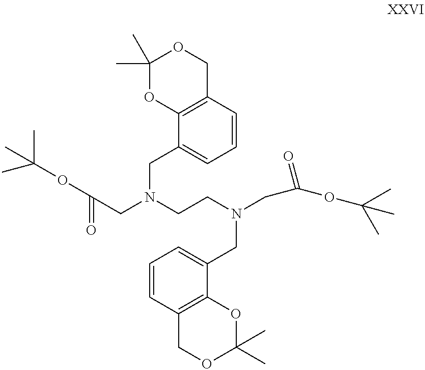

In yet another embodiment, the present invention provides a protected ligand precursor having s structure XXVII.

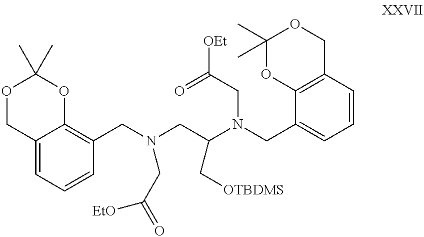

In yet another embodiment, the present invention provides a protected ligand precursor having structure XXVIII.

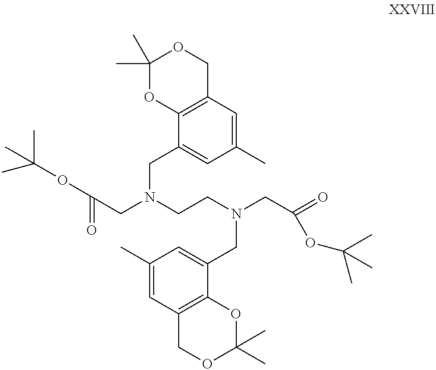

In another embodiment, the present invention provides a protected ligand precursor having structure XXIX.

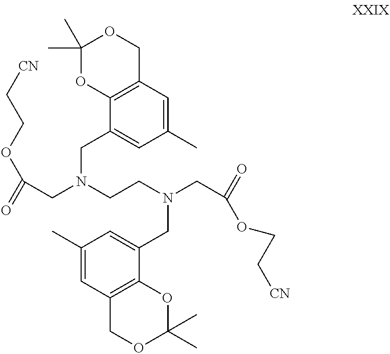

As mentioned above throughout this disclosure, no absolute or relative stereochemistry is intended to be shown for a structure, as in for example structures XX and XXIV, and the structures are intended to encompass all possible absolute and relative stereochemical configurations, unless specified otherwise. Thus, for example, structure XX depicts a compound in which no absolute or relative stereochemistry is intended to be shown. As such, structure XX is intended to represent a genus of compounds which includes the racemic compounds, single enantiomers, enantiomerically enriched compositions and mixtures of diastereomers.

In one embodiment, the present invention provides a medical formulation comprising the contrast enhancement agent having structure I. In yet another embodiment, the present invention provides a medical formulation comprising the contrast enhancement agent having structure II. In another embodiment, the medical formulations provided by the present invention comprise at least one structure selected from structures III, IV, V, VI, VII and VIII. The contrast enhancement agents provided by the present invention are suitable for use as imaging agents for magnetic resonance (MR) screening of human patients for various pathological conditions. As will be appreciated by those of ordinary skill in the art, MR imaging has become a medical imaging technique of critical importance to human health. In one embodiment, the present invention provides a method for increasing the emitted signal, and thus obtaining in vivo differentiation of tissues in an organism by administering a contrast enhancement agent of the present invention to a living subject and conducting magnetic resonance imaging of the subject. In one embodiment, the contrast enhancement agent provided by the present invention includes an iron chelate wherein the iron is paramagnetic. Contrast enhancement agents provided by the present invention comprising a paramagnetic iron center are believed to be more readily excreted by human patients and by animals and as such are more rapidly and completely cleared from the patient following the magnetic resonance imaging procedure. In addition, the contrast enhancement agents provided by the present invention may enable the administration of lower levels of the contrast enhancement agent to the patient relative to know contrast enhancement agents without sacrificing image quality. Thus, in one embodiment, useful MR contrast enhancement using the contrast enhancement agent of the present invention is achieved at lower dosage level in comparison with known MR contrast agents. In an alternate embodiment, the contrast enhancement agents provided by the present invention may administered to a patient at a higher dosage level in comparison with known MR contrast agents in order to achieve a particular result. Higher dosages of the contrast enhancement agents of the present invention may be acceptable in part because of the enhanced safety of such iron based contrast enhancement agents, and improved clearance of the contrast enhancement agent from the patient following the imaging procedure. In one embodiment, contrast enhancement agent is administered in a dosage amount corresponding to from about 0.001 to about 5 millimoles per kilogram weight of the patient. As will be appreciated by those of ordinary skill in the art, contrast enhancement agents provided by the present invention may be selected and/or further modified to optimize the residence time of the contrast enhancement agent in the patient, depending on the length of the imaging time required.

In one embodiment, the contrast enhancement agent according to the present invention may be used for imaging the circulatory system, the genitourinary system, hepatobiliary system, central nervous system, for imaging tumors, abscesses and the like. In another embodiment, the contrast enhancement agent of the present invention may also be useful to improve lesion detectability by MR enhancement of either the lesion or adjacent normal structures.

The contrast enhancement agent may be administered by any suitable method for introducing a contrast enhancement agent to the tissue area of interest. The medical formulation containing the contrast enhancement agent is desirably sterile and is typically administered intravenously and may contain various pharmaceutically acceptable agents, which promote the dispersal of the MR imaging agent. In one embodiment, the medical formulation provided by the present invention is an aqueous solution. In one embodiment, the MR imagining agent may be administered to a patient in an aqueous formulation comprising ethanol and the contrast enhancement agent. In an alternate embodiment, the MR imagining agent may be administered to a patient as an aqueous formulation comprising dextrose and the contrast enhancement agent. In yet another embodiment, the MR imagining agent may be administered to a patient as an aqueous formulation comprising saline and the contrast enhancement agent.

In addition to being useful as MR imaging agents and as probes for determining the suitability of a given iron chelate compound for use as a MR imaging agent, the contrast enhancement agents provided by the present invention may also, in certain embodiments, possess therapeutic utility in the treatment of one or more pathological conditions in humans and/or animals. Thus, in one embodiment, the present invention provides a contrast enhancement agent having structure I, which is useful in treating a pathological condition in a patient. In an alternate embodiment, the present invention provides a contrast enhancement agent having structure II, which is useful in treating a pathological condition in a patient.

Those skilled in the art will appreciate that iron chelate compounds falling within the scope of generic structure I may under a variety of conditions form salts which are useful as MR imaging agents, probes for the discovery and development of imaging agents, and/or as therapeutic agents. Thus, the present invention provides a host of novel and useful iron chelate compounds and their salts.

The contrast enhancement agent of the present invention may be prepared by a variety of methods including those provided in the experimental section of this disclosure. For example, stoichiometric amounts of the metal ion and the metal chelating ligand may be admixed in a solution with an appropriate adjustment of pH, if necessary. The contrast enhancement agent may be isolated by conventional methods such as crystallization, chromatography, and the like, and admixed with conventional pharmaceutical carriers suitable for pharmaceutical administration.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

### EXAMPLES

### Method 1

### Preparation of Diamine Compound 1

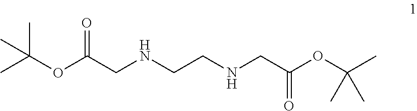

A solution of tert-butylbromoacetate (3.41 g, 17.47 mmol) in 5 milliliters (mL) of dimethyl formamide (DMF) was added over a 30 minute period to a solution of ethylene diamine (1.05 g, 17.47 mmol) in anhydrous dimethylformamide (30 mL) at 0° C. via a syringe pump. The reaction mixture was allowed to stand for about 2 h. At the end of the stipulated time the reaction mixture was analyzed by liquid chromatography mass spectrometry (LC-MS). The LC-MS analysis indicated the presence of a statistical mixture of alkylated products including mono, bis, bis′, tri, and tetrasubstituted products. The reaction mixture was then concentrated under reduced pressure and purified by C-18 reversed phase chromatography. The collected fractions containing the diamine compound 1 were combined and evaluated by LC-MS, m/z=289 [M+H]+.

### Method 2

### Preparation of Aldehyde Compound 2

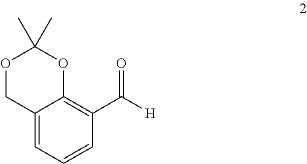

3-Bromosalicyl alcohol isopropylidene acetal (5.05 g, 22.1 mmol) was prepared as using the method described in Meier C. et al. Eur J. Org. Chem. 2006, 197. n-BuLi in hexanes (8.31 mL, 20.77 mmol) was diluted with 30 mL of anhydrous tetrahydrofuran (THF) and cooled to −75° C. A solution of 3-bromosalicyl alcohol isopropylidene acetal in 15 mL anhydrous THF was then added over a period of 1.5 h, while maintaining the internal reaction temperature at or below −70° C. in an acetone/dry ice bath. Following the addition of the 3-bromosalicyl alcohol isopropylidene acetal, the reaction mixture was stirred for an additional 30 min while maintaining the temperature at or below −70° C. At the end of 30 min anhydrous DMF (1.62 mL, 20.77 mmol) was added to the reaction mixture over a period of 30 sec. The reaction mixture was allowed to re-equilibrate to −70° C., and then warmed to 0° C. The reaction mixture was then quenched by the addition of methanol (30 mL), and was poured into saturated aqueous NaHCO₃, and then extracted with dichloromethane (3×75 mL). The combined organic extracts were dried over MgSO₄, filtered, and concentrated under reduced pressure to provide a yellow oil that solidified on standing under high vacuum. The crude material was purified by flash chromatography (SiO₂, 40 g column, isocratic, 10% EtOac-hexanes, 254 and 327 nm) to afford the aldehyde compound, 2, as a pale yellow solid, m/z=195 [M+3H]+.

### Example 1

### Preparation of Protected Ligand Precursor XXVI

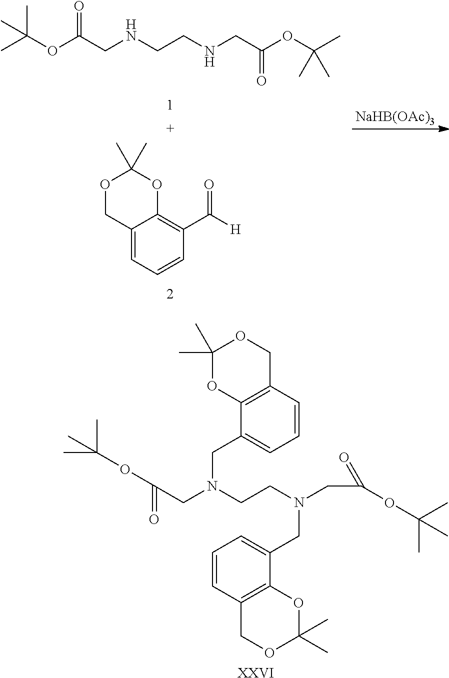

Diamine 1 (0.1 g, 0.35 mmol) and aldehyde 2 (0.13 g, 0.69 mmol) were dissolved in 1,2 dichloroethane (3.5 mL). Sodium acetoxyborohydride (0.33 g, 1.56 mmol) was then added to stirred reaction mixture and stirring was continued overnight. Reaction progress was monitored by LC-MS. The reaction mixture was diluted with saturated sodium bicarbonate solution and dichloromethane (10 mL). The aqueous and organic layers were separated and the aqueous layer was extracted with dichloromethane (3×25 mL). The combined organic layers were washed with saturated aqueous sodium bicarbonate, (2×25 mL), brine (2×25 mL), dried over MgSO₄ and filtered. The filtrate was concentrated under reduced pressure to provide the crude product XXVI as a pale yellow oil which was purified by flash chromatography (SiO₂, 12 g) using the following gradient program at 30 mL/min: 100% hexanes for 3 column volumes, then ramp to 35% EtOAc-hexanes over 20 column volumes, finally holding at 35% EtOAc-hexanes for 5 column volumes. The column eluant was monitored at 289 nm and fractions containing purified XXVI were pooled and concentrated under reduced pressure. Protected ligand precursor XXVI was obtained as a colorless oil that was further dried under high vacuum, m/z=642 [M+H]+.

### Example 2

### Preparation of Ligand Having Idealized Structure XV

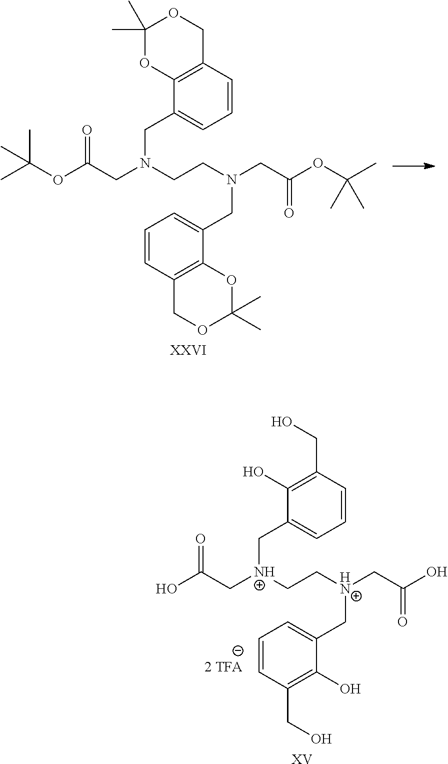

A mixture of dioxane (0.71 mL) and water (0.36 mL) were added to protected ligand precursor XXVI (0.11 g, 0.18 mmol) followed by the addition of 4M HCl in dioxane (0.71 mL). The reaction mixture was heated to 72° C. for 2 h and progress of the deprotection was monitored by LC-MS to ensure complete deprotection. The reaction mixture was then neutralized using a stoichiometric amount of 4M NaOH to pH 6. The mixture was concentrated under reduced pressure to provide a yellow foam that was analyzed by LC-MS and shown to contain the desired ligand, as well as other components. The crude product was purified by preparative high performance liquid chromatography (HPLC) on C18 functionalized silica gel (10×100 mm waters xTerra Prep C18 5 um) using the following gradient program at 9 mL/min: 2% MeCN-water containing 0.05% TFA for 0.5 minutes, then ramp to 60% MeCN-water containing 0.05% TFA over 14.5 minutes, finally holding at 60% MeCN-water containing 0.05% TFA for 3 minutes. The column eluant was monitored at 285 nm and the fractions containing the pure XV were pooled and concentrated under reduced pressure to provide the ligand XV, as a colorless oil, m/z=449 [M+H]+.

### Example 3

### Preparation of FeHBED(OH)₂ IV

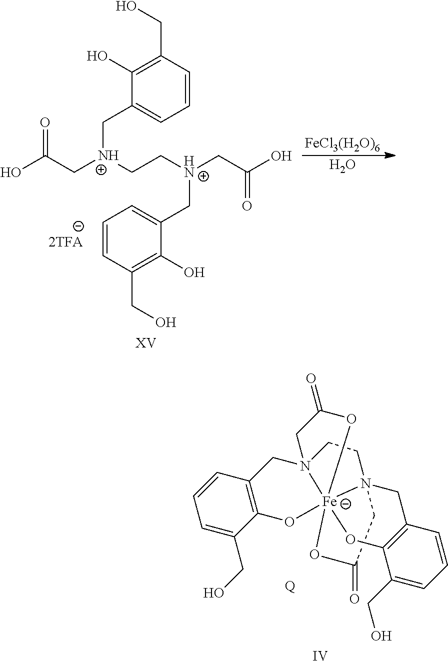

Ligand XV (4.5 mg, 7.0 mmol) was dissolved in deionized water (1.0 mL). To the resultant clear solution was added 1.5 mg of FeCl₃.6H₂O (6 mmol) dissolved in deionized water (100 mL) to form a dark red solution that was then quenched with NaHCO₃ (300 uL, 0.1M). The reaction mixture was passed through a Sephadex G-10 column, eluting with deionized water to afford iron chelate IV (also referred to as FeHBED(OH)2) wherein Q is a sodium cation as a clear red solution, m/z=501 [M+H]+, 524 [M+Na]+.

### Method 3

### Preparation of Aldehyde 5

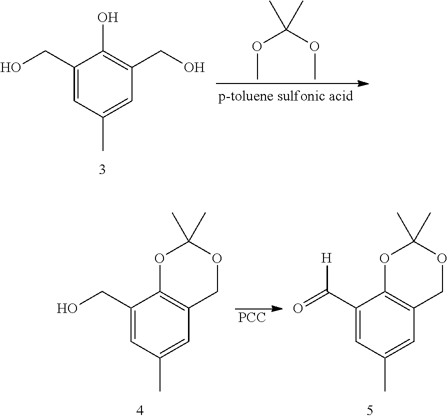

Aldehyde 5 was prepared according to the procedure given in Koskinen, A. M. P.; Abe, A. M. M.; Helaja, J. Org. Lett. 2006, 8, 20, 4537 which is incorporated herein by reference.

### Example 4

### Preparation of Protected Ligand Precursor XXVIII

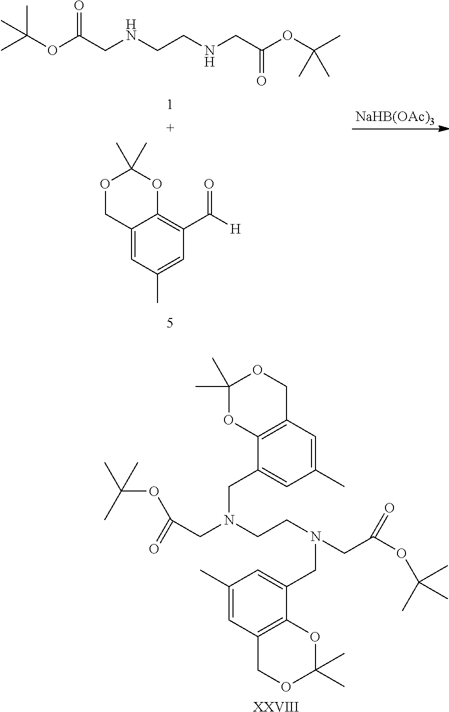

Diamine 1 (0.1 g, 0.35 mmol) and aldehyde 5 (0.14 g, 0.69 mmol) were dissolved in 1,2 dichloroethane (3.5 mL) followed by the addition of sodium acetoxyborohydride (0.33 g, 1.56 mmol). The reaction mixture was stirred at room temperature overnight and completion of the reaction was confirmed by LC-MS. The reaction mixture was diluted with saturated sodium bicarbonate solution and dichloromethane (10 mL). The aqueous and organic layers were separated and the aqueous layer was extracted with dichloromethane (3×25 mL). The combined organic layers were washed with saturated aqueous sodium bicarbonate, (2×25 mL), brine (2×25 mL), dried over MgSO₄ and filtered. The filtrate was concentrated under reduced pressure to provide the crude product as a pale yellow oil which was purified by flash chromatography (SiO₂, 12 g) using the following gradient program at 30 mL/min: 100% hexanes for 3 column volumes, then ramp to 35% EtOAc-hexanes over 20 column volumes, finally holding at 35% EtOAc-hexanes for 5 column volumes. The column eluant was monitored at 289 nm and fractions containing the purified protected ligand precursor XXVIII were pooled and concentrated under reduced pressure to yield XXVIII, as a colorless oil, m/z=669 [M+H]+.

### Example 5

### Preparation of Ligand Having Idealized Structure XIV

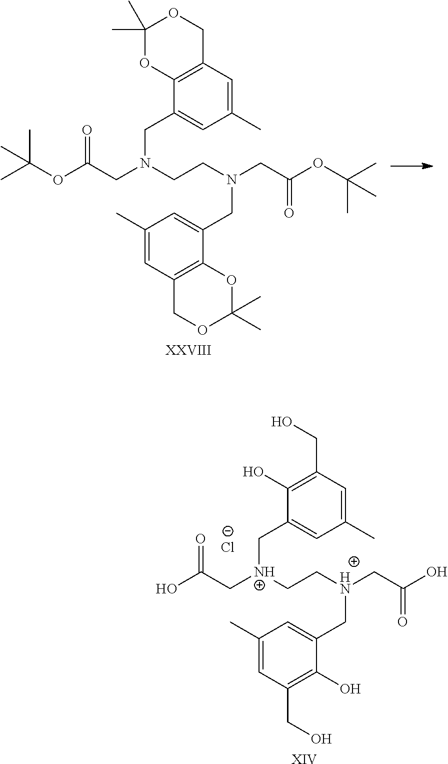

Dioxane (0.88 mL) and water (0.44 mL) were added to protected ligand precursor XXVIII (0.15 g, 0.22 mmol) followed by the addition of 4M HCl in dioxane (0.88 mL). The reaction mixture was allowed to stir at room temperature overnight and was then heated for about 90 min at 72° C. Complete deprotection of protected ligand precursor XXVIII was confirmed by LC-MS. The reaction mixture was then concentrated under reduced pressure and further dried under high vacuum to provide ligand XV as a white solid, m/z=477 [M+H]+. It should be noted that a small degree of decomposition (˜5-10%) was observed on concentration of the product mixture.

### Example 6

### Preparation of FeHBED(Me)₂(OH)₂ III

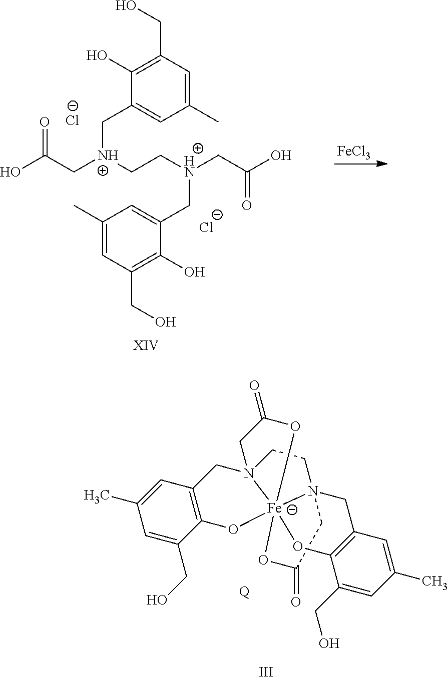

Deionized water (1.5 mL) was combined with ligand XIV (5.0 mg, 10 mmol) and FeCl₃.6H₂O (2.2 mg, 8.1 mmol) to afford a cloudy purple mixture. An aqueous solution of NEt₃HCO₃ (0.5 mL, 0.1 M) was then added to neutralize the reaction mixture, and afforded a clear, dark purple solution containing iron chelate III, as confirmed by LC-MS. The mixture was stirred for 12 h and then passed through a Sephadex G-10 plug, eluting with deionized water followed by a wash with diethyl ether (2×2 mL) to afford a purple solution which was concentrated under reduced pressure. The resulting purple solid was washed with CH₃CN (2×1 mL) and dried in-vacuo to give iron chelate III, wherein the charge balancing counterion Q was triethylammonium, as a purple solid, m/z=530 [M+2H]+.

### Method 4

### Preparation of Protected Diamine 6

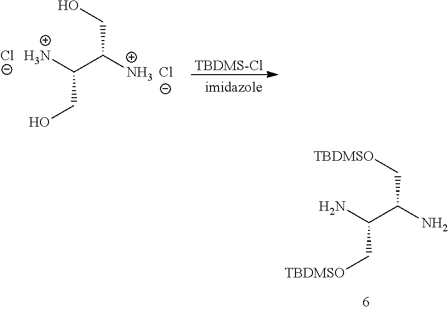

To a solution of 2,3-diamino butane-1,4-diol bishydrochloride (1.0 g, 5.8 mmol) and having the absolute stereochemistry shown, in dichloromethane (52 mL) was added imidazole (1.7 g, 25.9 mmol) followed by t-butyldimethylsilyl chloride (TBDMS-Cl, 1.6 g, 10.6 mmol). The reaction mixture was stirred overnight, and then quenched with saturated aqueous potassium carbonate. The aqueous and organic layers were separated. The aqueous layer was extracted with dichloromethane (3×25 mL) and the combined organic layers were washed with saturated aqueous potassium carbonate solution, (2×25 mL), brine, dried over MgSO₄ and filtered. The filtrate was concentrated under reduced pressure to provide the crude protected diamine 6 as a crystalline solid which was purified by flash chromatography on normal phase silica gel (40 gram column) using the following gradient program at 40 mL/min: 100% dichloromethane w/0.5% triethylamine for 2 column volumes, then ramp to 20% MeOH-dichloromethane each w/0.5% triethylamine over 20 column volumes, finally holding at 20% MeOH-dichloromethane each w/0.5% triethylamine for 3 column volumes. The column eluant was monitored at 230 nm and the fractions containing the pure product were pooled and concentrated under reduced pressure. Drying in-vacuo afforded protected diamine 6 having the absolute stereochemistry shown as a pale yellow oil, m/z=349 [M+H]+.

### Method 5

### Preparation of Protected Salicyl Aldehyde 7

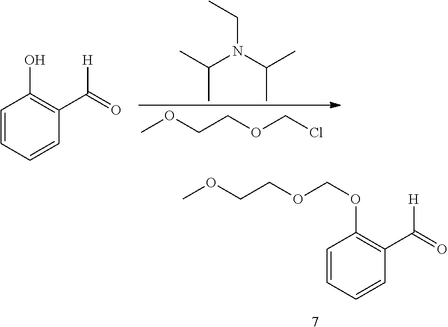

The protected aldehyde 7 was prepared analogously to procedures described in Breslow, R.; Schephartz, A. JACS, 1987, 109, 1814 and Hinterman, L.; Masuo, R.; Suzuki, K. Org. Lett. 2008, 10, 21, 4859, which are incorporated herein by reference.

### Method 6

### Preparation of Bisimine 8

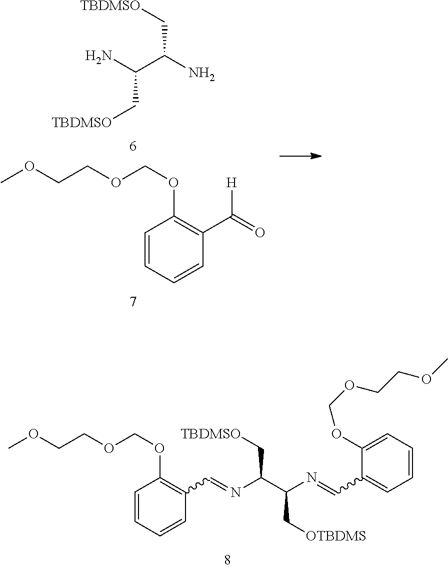

To a stirred suspension of protected diamine 6 (1.3 g, 3.73 mmol) in dichloromethane (10 mL), were added triethylamine (0.94 g, 9.32 mmol) and MgSO₄ (1.80 g, 14.9 mmol). After stirring for 1.5 h at room temperature a solution of aldehyde 7 (1.57 g, 7.46 mmol) in dichloromethane (5 mL) was added and the reaction mixture was stirred overnight. Because the bisimine product 8 was sensitive to hydrolysis, care was taken to exclude water from the workup and chromatographic steps. Thus, the reaction mixture was filtered and then concentrated under reduced pressure. The crude product was triturated with diethyl ether, filtered, and concentrated under reduced pressure to provide a yellow oil that was dried in vacuo. The complete conversion of starting materials to bisimine 8 was confirmed by NMR spectroscopy. ¹H NMR (CD₂Cl₂, 400 MHz) δ 0.06 (s, 6H), 0.11 (s, 6H), 0.93 (s, 18H), 3.36 (s, 6H), 3.54-3.58 (m, 4H), 3.65-3.70 (m, 2H), 3.75-3.80 (m, 2H), 3.81-3.84 (m, 4H), 4.07-4.13 (m, 2H), 5.32 (s, 4H), 7.03-7.09 9m, 2H), 7.20-7.25 (m, 2H), 7.37-7.43 (m, 2H), 8.01-8.07 (m, 2H) and 8.76 (s, 2H); ¹³C{¹H}NMR δ −5.49, 18.13, 25.69, 50.60, 66.83, 67.92, 71.59, 74.55, 93.70, 114.66, 121.65, 125.61, 127.42, 131.52, 156.77, and 157.86.

### Method 7

### Preparation of Diamine 9

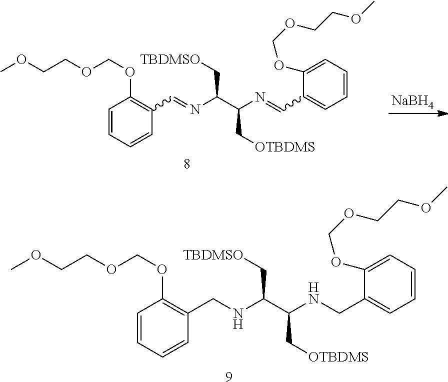

Bisimine 8 (1.38 g, 1.88 mmol) in methanol:dichloromethane (1.9 mL:7.5 mL) was treated with sodium borohydride (0.28 g, 7.5 mmol) at 0° C. The reaction mixture was stirred overnight room temperature and then diluted with saturated aqueous potassium carbonate. The aqueous and organic layers were separated and the aqueous layer was extracted with dichloromethane (3×25 mL) and the combined organic layers were washed with saturated aqueous sodium bicarbonate solution, (2×25 mL), brine (2×25 mL), dried over MgSO₄ and filtered. The filtrate was concentrated under reduced pressure to provide the crude product as a pale yellow oil which was purified by flash chromatography (SiO₂, 40 gram column) using the following gradient program at 60 mL/min: 100% dichloromethane w/0.5% triethylamine for 3 column volumes, then ramp to 5% MeOH-Dichloromethane each w/0.5% triethylamine over 20 column volumes, finally holding at 5% MeOH-Dichloromethane each w/0.5% triethylamine for 5 column volumes. The column eluant was monitored at 285 nm and the fractions containing the purified material were pooled, concentrated under reduced pressure and then dried in vacuo to yield purified diamine 9 as a colorless oil, m/z=738 [M+H]+.

### Example 7

### Preparation of Protected Ligand Precursor XXI

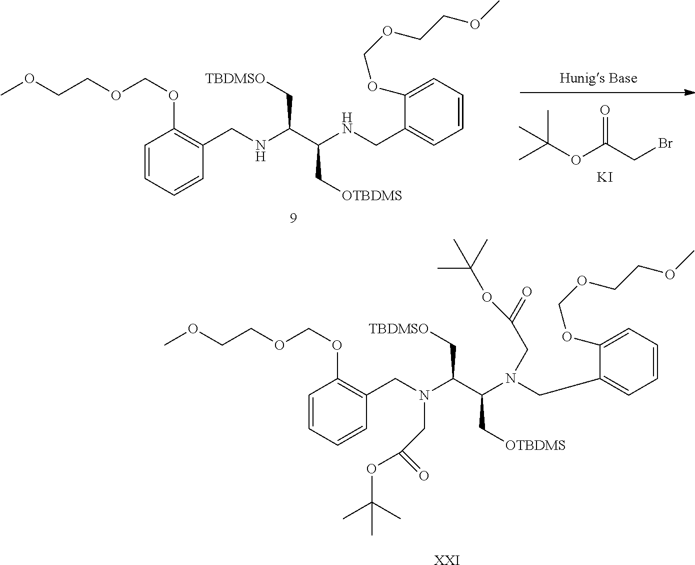

Hunig's base (0.20 g, 1.55 mmol) was added to a DMF (2.9 mL) solution of diamine 9 (0.29 g, 0.39 mmol) and the mixture was stirred for 30 min. In a separate vial, potassium iodide (0.19 g, 1.16 mmol) was dissolved in DMF (1 mL) and combined with tert-butyl bromoacetate (0.16 g, 0.82 mmol) and the mixture was stirred for 30 min and then added to the solution of diamine 9 and Hunig's base in DMF and the mixture was stirred overnight at 80° C. after which time LC-MS indicated that the reaction had proceeded to completion and also indicated the presence of minor impurities. The reaction mixture was concentrated under reduced pressure and the residue was dissolved in THF and filtered. The crude product was then dispersed onto SiO₂ and purified by flash chromatography (SiO₂, 12 gram column) using the following gradient program at 30 mL/min: 20% EtOAc-hexanes w/0.5% triethylamine for 3 column volumes, then ramp to 88% EtOAc-hexanes w/0.5% triethylamine over 20 column volumes, finally holding at 88% EtOAc-hexanes w/0.5% triethylamine for 5 column volumes. The column eluant was monitored at 277 nm and the purified material was pooled and concentrated under reduced pressure. Drying in vacuo provided the protected ligand precursor XXI as a colorless oil, m/z=966 [M+H]+.

### Example 8

### Preparation of FeHBED(OH′)₂ VII

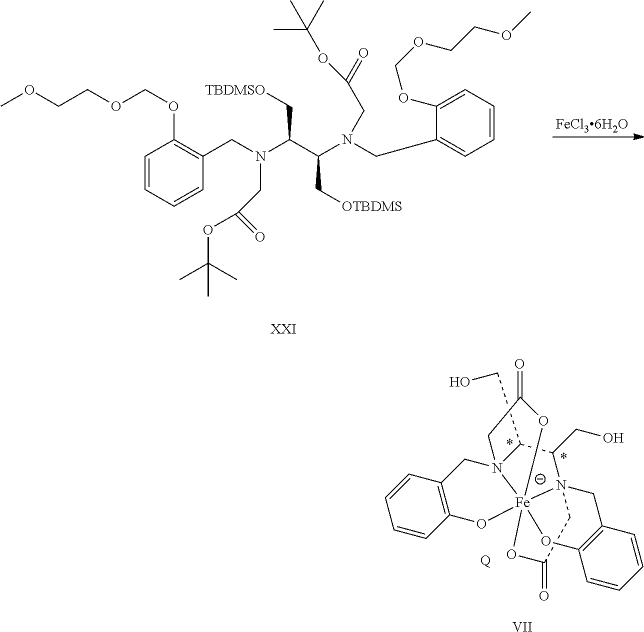

To a solution of the protected ligand precursor XXI (0.18 g, 0.18 mmol) in dioxane (1.22 mL) and deionized water (1.22 mL) was added FeCl₃.6H₂O (5.7 mg, 0.17 mmol). The reaction mixture was treated 4M HCl in dioxane (1.22 mL) and stirred at room temperature overnight and then heated to 75° C. in an oil bath for 2 hours. Completion of the reaction was confirmed by LC-MS analysis of a reaction mixture aliquot, which had been neutralized with saturated aqueous sodium bicarbonate. The reaction mixture was then cooled to 0° C. in an ice bath and quenched with aqueous sodium bicarbonate. The resultant mixture was diluted with deionized water (10 mL) and dichloromethane (10 mL). The aqueous and the organic layers were separated. The aqueous layer was washed with dichloromethane (3×25 mL) and the combined organic layers that were extracted with deionized water, (2×25 mL). The aqueous layers were combined and concentrated under reduced pressure (50 torr, 40° C., 30 min) to a reduced volume. The resultant red solution was filtered through a 30,000 molecular weight cut-off filter and lyophilized to afford iron chelate VII as a red solid having the same absolute stereochemistry at the centers marked with an asterisk (*) as shown in protected ligand precursor XXI, and wherein the charge balancing counterion Q is sodium cation. LC-MS analysis of the product iron chelate VII indicated a mixture of two diastereomers in a 65:35 ratio, m/z=502[M+H]+ with trace amounts of the free ligand corresponding to protected ligand precursor XXI.

### Method 8

### Preparation of Compound 10

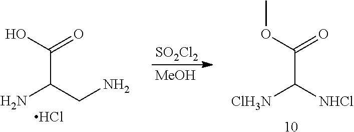

Thionyl chloride (31.7 g, 266.8 mmol) was added dropwise to a stirred suspension of 2,3-diaminopropionic acid monohydrochloride (5.0 g, 35.6 mmol) in methanol (75 mL) over a period of about 5 min. The reaction mixture was heated to about 80° C. for about 6 hours. At the end of the stipulated time, the reaction mixture was cooled and the volatiles were removed under reduced pressure to obtain compound 10 (6.8 g, 100%) as an off-white solid. ¹H NMR (MeOD): δ 4.51 (m, 1H), δ 3.96 (s, 3H), δ 3.53 (m, 2H).

### Method 9

### Preparation of Protected Aldehyde 11

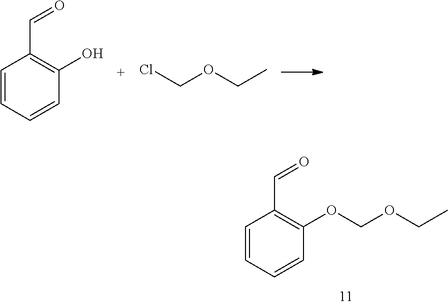

Diisopropylethylamine (8.64 g, 66.8 mmol) was added to a stirred solution of salicylaldehyde (5.83 g, 47.7 mmol) in dichloromethane (477 mL) at 0° C. in an ice-bath. The reaction mixture was allowed to stand for 1 hour and then chloromethoxyethane (4.74 g, 50.1 mmol) was added dropwise over a period of 5 minutes. The pale yellow reaction mixture was warmed to ambient temperature and stirred for 18 hours. The reaction mixture was diluted with saturated aqueous ammonium chloride (100 mL) and the layers were separated. The aqueous layer was extracted with dichloromethane (2×50 mL). The organic layers were combined and dried over MgSO₄ and filtered. The filtrate was concentrated under reduced pressure to afford the crude product as a yellow oil which was purified by column chromatography (SiO₂, hexanes to 1:9 ethyl acetate:hexanes) to afford protected aldehyde 11 as a nearly colorless oil, m/z=181 [M+H]+.

### Method 10

### Preparation of Bisimine 12

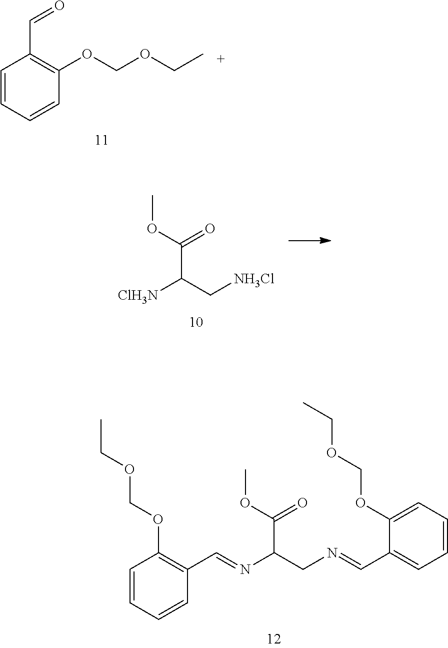

To a stirred solution of diamine 10 (2.69 g, 14.1 mmol) in anhydrous methylene chloride (50 mL) was added triethylamine (6.41 g, 63.4 mmol). The reaction mixture stirred for about 45 minutes. MgSO₄ (6.78 g, 56.3 mmol) was then added and the mixture was stirred for an additional 45 minutes. A solution of protected aldehyde 11 (5.15 g, 28.6 mmol) in methylene chloride (5 mL) was then added over a period of 2 min and the colorless mixture was stirred for 18 hours at ambient temperature. The yellow-orange reaction mixture was filtered and the filtrate was concentrated under reduced pressure to afford an oil. The oil was dissolved in methylene chloride and added with stirring to diethyl ether (250 mL) to afford a white precipitate (Et₃NHCl). The mixture was filtered and the filtrate concentrated under reduced pressure to afford bisimine 12 as a yellow-orange oil the structure of which was confirmed by NMR spectroscopy. ¹H NMR (CD₂Cl₂): δ 8.72 (s, 1H), δ 8.70 (s, 1H), δ 7.98 (dd, J=7.0 Hz, J=7.0 Hz, 1H), δ 7.91 (dd, J=7.0 Hz, J=7.0 Hz, 1H), 7.38 (m, 2H), δ 7.15 (t, J=8.0 Hz, 2H), δ 7.02 (m, 2H), δ 5.23 (s, 4H), δ 4.43 (m, 1H), δ 4.32 (m, 1H), δ 3.91 (m, 1H), δ 3.79 (s, 3H), δ 3.68 (m, 4H), δ 1.19 (t, J=7.0 Hz, 1H).

### Method 11

### Preparation of Diamine 13

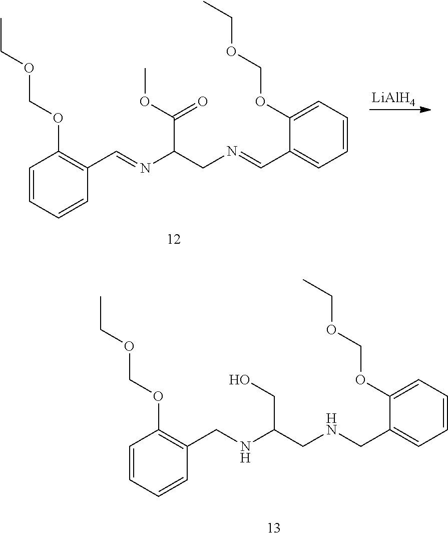

To a stirred solution of compound 12 (2.0 g, 4.52 mmol) in anhydrous tetrahydrofuran (50 mL) at 0° C. (ice-bath) was added lithium aluminum hydride (0.69 g, 18.1 mmol) was added in portions over a period of about 5 minutes. The resultant greenish-grey reaction mixture was warmed to ambient temperature and stirred for 18 hours. Deionized water (8-10 mL) was then added dropwise over a period of 5 minutes and the resultant mixture was stirred for 1.5 hours. The mixture was filtered and the filtrate was concentrated under reduced pressure to afford the crude product diamine 13 as a yellow oil which was purified by column chromatography (SiO₂, 99% methylene chloride:1% triethylamine to 94% methylene chloride:5% methanol:1% triethylamine) to obtain purified diamine 13 as a pale yellow oil, m/z=419 [M+H]+.

### Method 12

### Preparation of Diamine 14

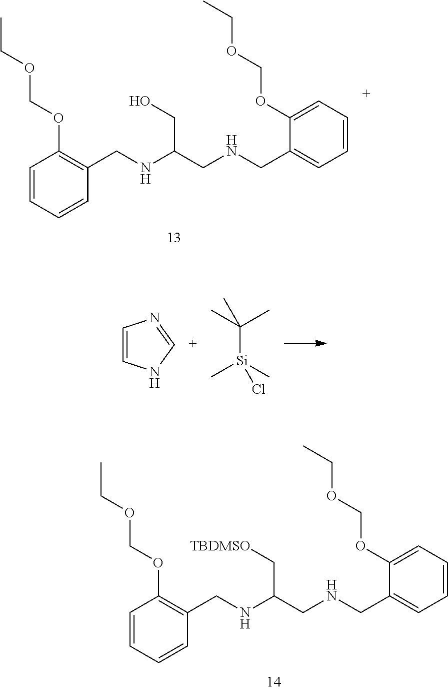

To a cooled (0° C.) stirred solution of diamine 13 (1.00 g, 2.39 mmol) in anhydrous dichloromethane (50 mL) was added imidazole (0.65 g, 9.56 mmol) and the mixture was stirred for 30 minutes after which time tert-butyldimethylsilyl chloride (0.38 g, 2.51 mmol) was added. The resulting pale yellow reaction mixture was warmed to ambient temperature and stirred for 18 hours. Saturated aqueous potassium carbonate (50 mL) was then added and the layers were separated. The aqueous layer was extracted with dichloromethane (2×25 mL), and the organic layers were combined and concentrated under reduced pressure to afford the crude product as a yellow oil. The crude product was purified by column chromatography (silica, hexanes to 1:9 ethyl acetate:hexanes) to afford purified diamine 14 (1.08 g, 85%) as a nearly colorless oil, m/z=533 [M+H]+.

### Example 9

### Preparation of Protected Ligand Precursor 6c

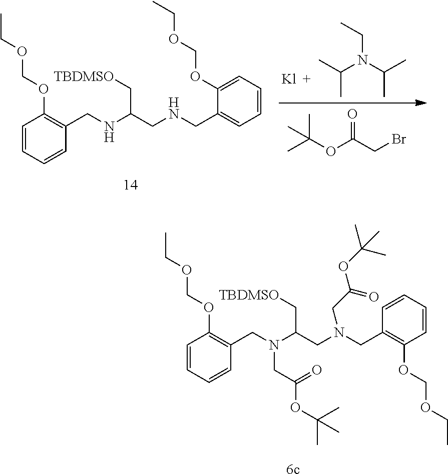

To a stirred solution of diamine 14 (1.08 g, 2.03 mmol) in N,N-dimethylformamide (20 mL) was added diisopropylethylamine (0.79 g, 6.08 mmol). Stirring was continued for 45 minutes, followed by the addition of a separately prepared solution of potassium iodide (1.35 g, 8.11 mmol) and tert-butylbromoacetate (0.83 g, 4.26 mmol) in N,N-dimethylformamide (5 mL). The resultant pale yellow reaction mixture was heated at 80° C. for 18 hours. The resultant reddish-brown product mixture was cooled to ambient temperature and concentrated under reduced pressure to afford the crude product as a dark oil which was subjected to column chromatography (SiO₂, hexanes to 1:9 ethyl acetate:hexanes) to afford purified protected ligand precursor 6c (0.88 g, 57%) as a pale yellow oil. m/z=762 [M+H]+F.

### Example 10

### Preparation of Ligand 4f

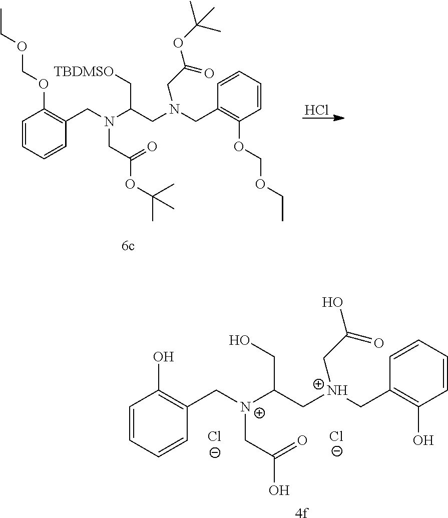

To a stirred solution of the protected ligand precursor 6c (0.88 g, 1.15 mmol) in acetonitrile (1 mL) was added 1 M aqueous hydrochloric acid (2 mL) and the reaction heated to 50° C. for 18 hours. The reaction mixture was neutralized with 5N sodium hydroxide (0.80 mL) to pH 7.1-7.3. The neutralized solution was concentrated under reduced pressure to obtain ligand 4f as an off white solid that was used without further purification, m/z=419 [M+H]+.

### Example 11

### Preparation of FeHBED(OH) VI

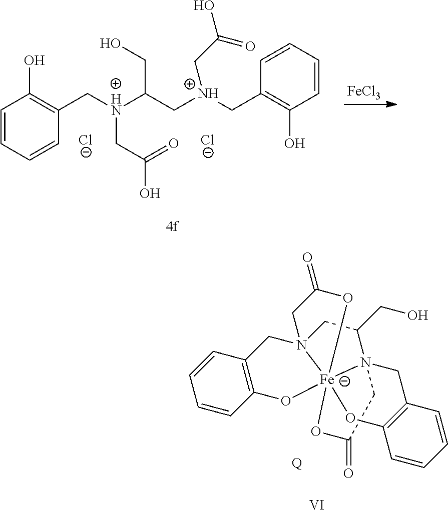

Ligand 4f (488 mg, 1.15 mmol) was dissolved in MeOH (7 mL) to provide a homogeneous colorless solution. An orange solution of FeCl₃ (132 mg, 81 mmol) dissolved in MeOH (3 mL) was added dropwise to the ligand solution to form a purple reaction mixture which was stirred for 10 minutes at ambient temperature. Hunig's base (NEtⁱPr₂, 300 μL, 1.7 mmol) was then added dropwise over a 5 minute period to afford homogeneous dark red solution having a pH of 6.5. The dark red solution was allowed to stir for 12 hours. Deionized water (5 mL) was added and the resultant mixture was extracted with Et₂O (3×15 mL). The aqueous layer was deposited atop a Sephadex G10 plug (2 g) and eluted with two portions (2×10 mL) of deionized water followed by two portions of MeOH (2×10 mL) to afford a homogeneous red solution. The clear red solution was lyophilized to provide the iron chelate VI, wherein the charge balancing counterion Q is the protonated form of NEtⁱPr₂, as a red solid (269 mg, 56% yield). LC-MS 472 m/z [M+H]+. UV-Vis (DI) λₘₐₓ=492 nm.

### Method 13

### Preparation of Bisimine 15

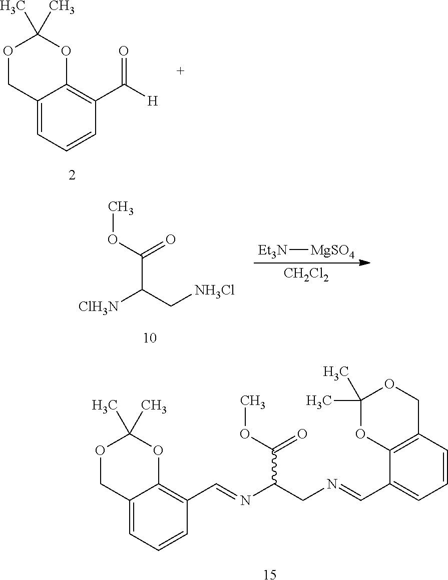

Triethylamine (2.38 g, 23.6 mmol) and MgSO₄ (2.52 g, 20.5 mmol) were added to a suspension of diamine bishydrochloride 10 (1.00 g, 5.23 mmol) in dichloromethane (15 mL) and the mixture was stirred for 1.5 hours at room temperature. A solution of the protected aldehyde 2 (2.04 g, 10.4 mmol) in dichloromethane (6 mL) was then added and the reaction mixture was stirred overnight at ambient temperature. As the desired bisimine product 15 was suspected of being highly susceptible to hydrolysis, care was taken to exclude water from the workup and chromatographic steps. The reaction mixture was filtered and concentrated under reduced pressure to provide bisimine 15 containing a small quantity of unreacted aldehyde as confirmed by NMR: ¹H NMR (CD₂Cl₂, 400 MHz) δ 1.50 (s, 3H), 1.51 (s, 3H), 1.58 (s, 6H), 3.81 (s, 3H), 3.92-4.00 (m, 1H), 4.33-4.41 (m, 1H), 4.45-4.51 (m, 1H), 4.85 (s, 4H), 6.92-6.97 (m, 2H), 7.02-7.08 (m, 2H), 7.84-7.88 (m, 1H), 7.92-7.96 (m, 1H), 8.69 (s, 1H), and 8.71 (s, 1H); ¹³C{¹H} NMR δ 24.38, 24.43, 24.73, 24.78, 46.20, 52.00, 60.62, 63.41, 73.51, 100.04, 100.15, 119.99, 120.01, 123.62, 124.00, 125.57, 125.79, 127.12, 127.57, 130.90, 150.94, 151.21, 158.63, 159.72, 171.48, and 188.59.

### Method 14

### Preparation of Diamine 16

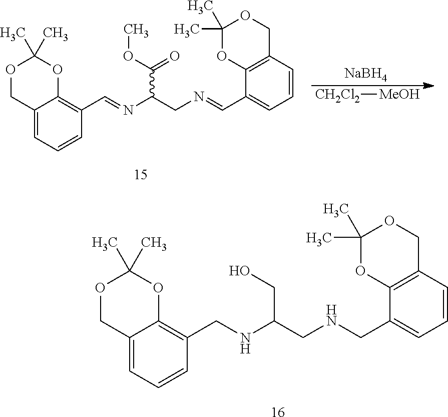

A solution of sodium borohydride (1.19 g, 31.4 mmol) in methanol (5.23 mL) was added dropwise via an additional funnel to a stirred solution of bisimine 15 (2.44 g, 5.23 mmol) in dichloromethane (20.9 mL) at 0° C. The reaction mixture was stirred overnight at room temperature and then diluted with saturated aqueous potassium carbonate. The aqueous and organic layers were separated. The aqueous layer was extracted with dichloromethane (3×25 mL) and the combined organic layers were washed with saturated aqueous sodium bicarbonate, (2×25 mL), and brine (2×25 mL), dried over MgSO₄ and filtered. The filtrate was concentrated under reduced pressure to provide the crude product diamine as a pale yellow oil which was purified by flash chromatography (SiO₂, 40 gram column) using the following gradient program at 60 mL/min: 100% dichloromethane w/0.5% triethylamine for 3 column volumes, then ramp to 5% MeOH-Dichloromethane each w/0.5% triethylamine over 20 column volumes, finally holding at 5% MeOH-Dichloromethane each w/0.5% triethylamine for 5 column volumes. The column eluant was monitored at 285 nm and fractions containing the purified product were pooled and concentrated under reduced pressure. Diamine 16 was obtained as a colorless oil that was dried under high vacuum, m/z=444 [M+H]+.

### Example 12

### Preparation of Protected Ligand Precursor XXX

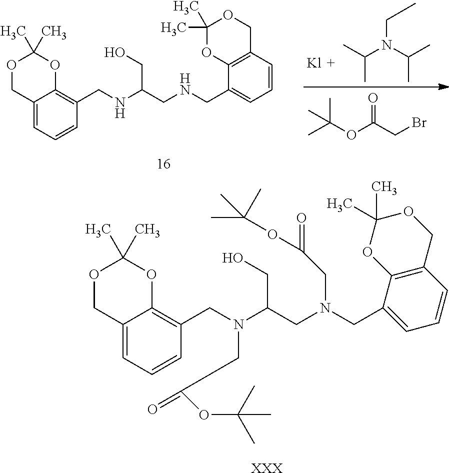

Diamine 16 was dissolved in DMF (7.5 mL). Hunig's base (0.49 g, 3.8 mmol) was added and the mixture was stirred for 30 minutes. In a separate vial, tert-butylbromoacetate (0.39 g, 2.0 mmol) was added to a DMF (2 mL) solution of potassium iodide (0.47 g, 2.9 mmol) and the mixture was stirred for about 30 minutes. The potassium iodide-tert-butylbromoacetate mixture was then added to the solution of diamine 16 and Hunig's base and the reaction mixture was stirred overnight at 80° C. The product mixture was analyzed by LC-MS which indicated that the reaction had proceeded to completion. The reaction mixture was concentrated under reduced pressure, and the residue was dissolved in THF and filtered. The filtrate was then adsorbed onto SiO₂ and subjected to column chromatography (SiO₂, 12 g column, 17.5% EtOAc-25% EtOAc:hexanes over 25 column volumes (CV) eluant was observed at 281 nm). The fractions containing purified product were combined, concentrated under reduced pressure and dried in vacuo to obtain protected ligand precursor XXX as a colorless oil, LCMS m/z=672 [M+H]+, 693 [M+Na]+.

### Example 13

### Preparation of Iron Chelate V

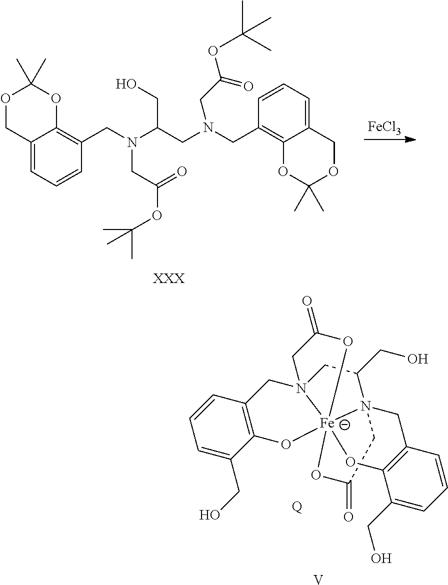

The protected ligand precursor XXX was dissolved in acetonitrile (1.38 mL) and water (0.17 mL) and FeCl₃ (3.6 mg, 22.6 μmol) was added followed by concentrated HCl (12 M, 172 μL). The reaction vessel was sealed and heated to 70° C. Progress of the reaction was monitored by LC-MS analysis of aliquots quenched with saturated aqueous sodium bicarbonate. After 4 hours, conversion of protected ligand precursor XXX to the product iron chelate appeared to be complete. The reaction mixture was then quenched by the addition of saturated aqueous sodium bicarbonate and concentrated to dryness under reduced pressure. The residue was dissolved in a minimal amount of water and filtered through a 5 μm nylon filter. The crude product was purified by preparative HPLC on C18 functionalized silica gel (10×100 mm waters xTerra Prep C18 5 μm) using the following gradient program at 9 mL/min: 100% water for 0.5 minutes, then ramp to 10% MeCN-water containing 0.05% TFA over 14.5 minutes, finally holding at 10% MeCN-water containing 0.05% TFA for 3 minutes. The column eluant was monitored at 494 nm and fractions containing the purified product iron chelate were pooled and concentrated under reduced pressure and dried under high vacuum to obtain the iron chelate V wherein Q is a sodium cation as a red solid, m/z=532 [M+H]+, 554 [M+Na]+. UV-Vis (DI) λₘₐₓ=494 nm

### Relaxivity Determinations

A stock solution having a concentration of 1 mM of the contrast enhancement agent was prepared in phosphate buffered saline (PBS) and the iron concentration was verified by elemental analysis. Separate 0.75 mM, 0.50 mM and 0.25 mM samples were prepared from the stock by dilution in PBS and the T₁ and T₂ relaxations times were recorded in triplicate for each using sample on a Bruker Minispec mq60 instrument (60 MHz, 40° C.). The relaxivities (r₁ and r₂) were obtained as the gradient of 1/Tₓ (x=1,2) plotted against Fe chelate concentration following linear least squares regression analysis. Data for contrast enhancement agents having structures III, IV, V, VI, VII, and VII, and a non-hydroxylated control contrast enhancement agent. Data are gathered in Table 8 below and illustrate the surprising effect of hydroxylation on the relaxivities exhibited by the contrast enhancement agents provided by the present invention relative to the control sample.

TABLE 8
Relaxivities Of Representative Contrast Enhancement Agents

| | | No. Hydroxy | r₁ | r₂ |
|---|---|---|---|---|
| | Chelate Structure | Groups | (mM⁻¹.s⁻¹) | (mM⁻¹.s⁻¹) |
| Control | 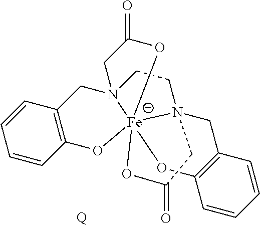 | 0 | 0.5 | 0.5 |
| III | 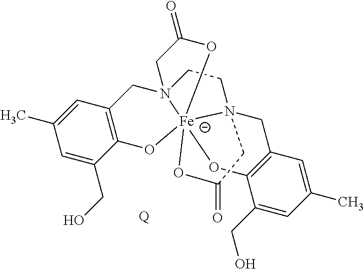 | 2 | 0.9 | 1.0 |
| IV | 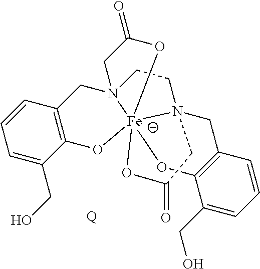 | 2 | 0.8 | 1.0 |
| V | 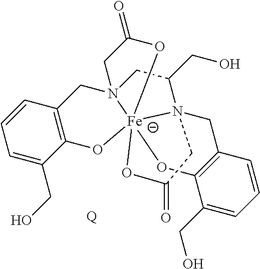 | 3 | 0.9 | 1.0 |
| VI | 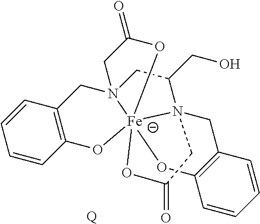 | 1 | 1.1 | 1.5 |
| VII | 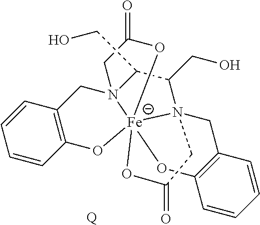 | 2 | 1.0 | 1.1 |
| VIII | 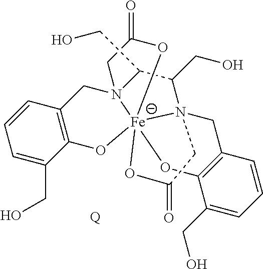 | 4 | — | — |

The foregoing examples are merely illustrative, serving to illustrate only some of the features of the invention. The appended claims are intended to claim the invention as broadly as it has been conceived and the examples herein presented are illustrative of selected embodiments from a manifold of all possible embodiments. Accordingly, it is the Applicants' intention that the appended claims are not to be limited by the choice of examples utilized to illustrate features of the present invention. As used in the claims, the word “comprises” and its grammatical variants logically also subtend and include phrases of varying and differing extent such as for example, but not limited thereto, “consisting essentially of” and “consisting of.” Where necessary, ranges have been supplied; those ranges are inclusive of all sub-ranges there between. It is to be expected that variations in these ranges will suggest themselves to a practitioner having ordinary skill in the art and where not already dedicated to the public, those variations should where possible be construed to be covered by the appended claims. It is also anticipated that advances in science and technology will make equivalents and substitutions possible that are not now contemplated by reason of the imprecision of language and these variations should also be construed where possible to be covered by the appended claims.

What is claimed is:

1. A protected ligand precursor having structure XX 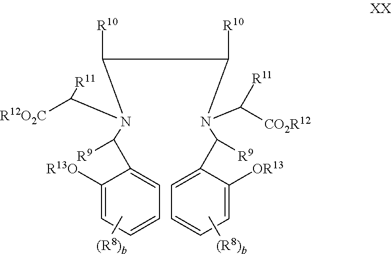
wherein R⁸ is independently at each occurrence a protected hydroxy group, a protected C₁-C₃ hydroxyalkyl group, or a C₁-C₃ alkyl group, and b is 0-4; R⁹-R¹¹ are independently at each occurrence hydrogen, a protected C₁-C₃ hydroxyalkyl group, or a C₁-C₃ alkyl group, with the proviso that at least one of R⁸-R¹¹ is a protected hydroxy group or a protected C₁-C₃ hydroxyalkyl group; and R¹² and R¹³ are independently at each occurrence a protecting group selected from the group consisting of C₁-C₃₀ aliphatic radicals, C₃-C₃₀ cycloaliphatic radicals, and C₂-C₃₀ aromatic radicals.

2. The protected ligand precursor according to claim 1, wherein R¹² is independently at each occurrence an ethyl group, a trichloroethyl group, a beta-cyanoethyl group, a trimethylsilyl ethyl group, or a tertiary butyl group.

3. The protected ligand precursor according to claim 1, wherein R¹² is a trimethylsilyl group.

4. The protected ligand precursor according to claim 1, wherein R¹² is a t-butyldimethylsilyl group.

5. The protected ligand precursor according to claim 1, wherein R¹² is an ethyl group.

6. The protected ligand precursor according to claim 1, wherein R¹³ is a THP group.

7. The protected ligand precursor according to claim 1, wherein R¹³ is a methoxthyethoxymethyl group.

8. The protected ligand precursor according to claim 1, wherein R¹³ is a t-butyldimethylsilyl group.

9. The protected ligand precursor according to claim 1, wherein R¹³ is a trimethylsilyl group.

10. The protected ligand precursor according to claim 1, having structure XXI

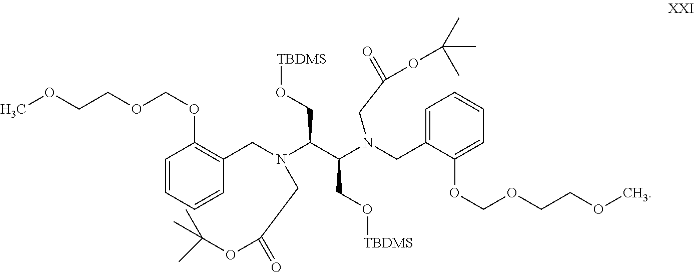

11. The protected ligand precursor according to claim 1, having structure XXII

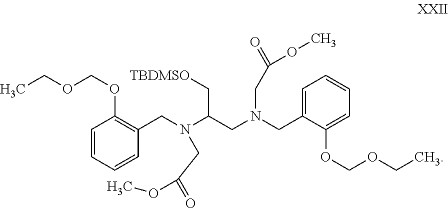

12. The protected ligand precursor according to claim 1, having structure XXIII

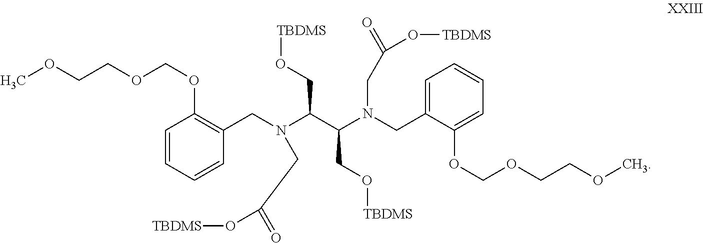

13. The protected ligand precursor according to claim 1, which is a racemate, a single enantiomer, an enantiomerically enriched composition, or a mixture of diastereomers.

14. A protected ligand precursor having structure XXIV 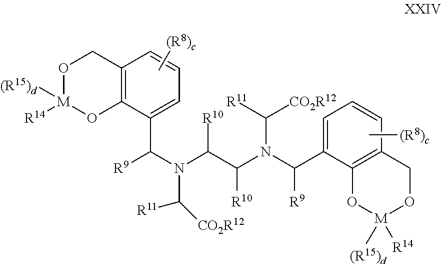
wherein R⁸ is independently at each occurrence a protected hydroxy group, a protected C₁-C₃ hydroxyalkyl group, or a C₁-C₃ alkyl group; R⁹-R¹¹ are independently at each occurrence hydrogen, a protected C₁-C₃ hydroxyalkyl group, or a C₁-C₃ alkyl group; R¹² is independently at each occurrence a protecting group selected from the group consisting of C₁-C₃₀ aliphatic radicals, C₃-C₃₀ cycloaliphatic radicals, and C₂-C₃₀ aromatic radicals; R¹⁴ and R¹⁵ are independently at each occurrence a C₁-C₁₀ alkyl group, a C₁-C₁₀ alkoxy group, or aryl group; M is independently at each occurrence a B, Si or carbon; c is 0-3; and d is 0 or 1.

15. The protected ligand precursor according to claim 14, wherein R¹² is independently at each occurrence an ethyl group, a trichloroethyl group, a beta-cyanoethyl group, trimethylsilyl ethyl group, or a tertiary butyl group.

16. The protected ligand precursor according to claim 14, wherein R¹² is a trimethylsilyl group.

17. The protected ligand precursor according to claim 14, wherein R¹² is a t-butyldimethylsilyl group.

18. The protected ligand precursor according to claim 14, having structure XXV

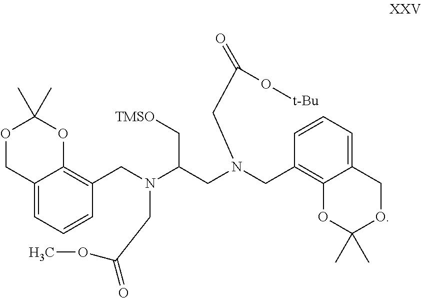

19. The protected ligand precursor according to claim 14, having structure XXVII [chemical structure]
